SEQUENCE LISTING (22 sequences)

SEQ ID NO: 1 - protein, 330 aa, Artificial Sequence [sequence omitted]

SEQ ID NO: 2 - protein, 326 aa, Artificial Sequence
  Human IgG2 Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Artificial Sequence
  Human IgG3 Heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Artificial Sequence
  Human IgG4 Heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 8 aa, Artificial Sequence
  OVA peptide
SIINFEKL

SEQ ID NO: 6 - protein, 282 aa, Artificial Sequence
  Human B7-H4 amino acid sequence with signal sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 7 - protein, 258 aa, Artificial Sequence
  Human B7-H4 amino acid sequence without predicted signal sequence
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 8 - protein, 236 aa, Artificial Sequence
  Human B7-H4 Extracellular domain amino acid sequence
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASL

SEQ ID NO: 9 - protein, 6 aa, Artificial Sequence
  278M1 Heavy chain CDR1
TSYYMH

SEQ ID NO: 10 - protein, 17 aa, Artificial Sequence
  278M1 Heavy chain CDR2
YVDPFNGGTSYNQKFKG

SEQ ID NO: 11 - protein, 7 aa, Artificial Sequence
  278M1 Heavy chain CDR3
FIAGFAN

SEQ ID NO: 12 - protein, 6 aa, Artificial Sequence
  278M1 Heavy chain CDR3
IAGFAN

SEQ ID NO: 13 - protein, 11 aa, Artificial Sequence
  278M1 Light chain CDR1
KASQDIKSYLS

SEQ ID NO: 14 - protein, 7 aa, Artificial Sequence
  278M1 Light chain CDR2
YATSLAD

SEQ ID NO: 15 - protein, 9 aa, Artificial Sequence
  278M1 Light chain CDR3
LQHGESPYT

SEQ ID NO: 16 - protein, 8 aa, Artificial Sequence
  278M1 Light chain CDR3
LQHGESPY

SEQ ID NO: 17 - protein, 115 aa, Artificial Sequence
  278M1 Heavy chain variable region
QVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSA

SEQ ID NO: 18 - protein, 107 aa, Artificial Sequence
  278M1 Light chain variable region
DIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIK

SEQ ID NO: 19 - protein, 458 aa, Artificial Sequence
  278M1 Heavy chain amino acid sequence
  SIGNAL (1)..(19): signal sequence
MKHLWFFLLLVAAPRWVLSQVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 439 aa, Artificial Sequence
  278M1 Heavy chain amino acid sequence without signal sequence
QVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 21 - protein, 233 aa, Artificial Sequence
  278M1 Light chain amino acid sequence
  SIGNAL (1)..(19): signal sequence
MKHLWFFLLLVAAPRWVLSDIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 214 aa, Artificial Sequence
  278M1 Light chain amino acid sequence without signal sequence
DIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2019/0048094 A1
(43) Pub. Date: Feb. 14, 2019

(54) REDIRECTING IMMUNE RESPONSES

(71) Applicant: OncoMed Pharmaceuticals, Inc., Redwood City, CA (US)

(72) Inventors: Austin L. GURNEY, San Francisco, CA (US); Cecile CHARTIER-COURTAUD, Palo Alto, CA (US)

(73) Assignee: OncoMed Pharmaceuticals, Inc., Redwood City, CA (US)

(21) Appl. No.: 16/079,180

(22) PCT Filed: Feb. 24, 2017
(86) PCT No.: PCT/US2017/019272

§ 371 Date: Aug. 23, 2018

Related U.S. Application Data
(60) Provisional application No. 62/299,164, filed on Feb. 24, 2016.

(51) Int. Cl. C07K 16/30; C07K 14/005; A61K 39/395; A61K 45/06

(52) U.S. Cl. CPC C07K 16/30; C07K 14/005; A61K 39/39558; A61K 2039/505; C07K 2319/75; C07K 2317/77; C07K 2317/73; A61K 45/06

(57) ABSTRACT

Agents that specifically bind tumor-associated antigens (TAA) and comprise an exogenous polypeptide or peptide that can be presented by a tumor cell are disclosed. The TAA-binding agents may include antibodies and/or bispecific agents. Also disclosed are methods of using the agents for redirecting an existing immune response against tumor cells and/or treatment of diseases such as cancer.

### CROSS-REFERENCE TO RELATED APPLICATIONS

This application claims the priority benefit of U.S. Provisional Application No. 62/299,164, filed Feb. 24, 2016, which is hereby incorporated by reference herein in its entirety.

### FIELD OF THE INVENTION

The present invention generally relates to agents that bind tumor-associated antigens and comprise at least one T-cell epitope, wherein the agent is internalized and the T-cell epitope is presented on the surface of a cell. The invention provides methods of using the agents for the modulation of immune responses and/or the treatment of diseases such as cancer.

### BACKGROUND OF THE INVENTION

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. Immunotherapy may include agents and methods to induce or enhance specific immune responses or to inhibit or reduce specific immune responses.

The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Cancer/tumor immunotherapy focuses on the development of new and novel agents that can activate and/or boost the immune system to achieve a more effective attack against tumor cells resulting in increased killing of tumor cells and/or inhibition of tumor growth.

### BRIEF SUMMARY OF THE INVENTION

The present invention provides novel agents, including, but not limited to, antibodies, homodimeric molecules, or heterodimeric molecules that modulate the immune response. The agents include antibodies that specifically bind tumor-associated antigens and redirect an existing immune response to target a tumor cell.

In one aspect, the invention provides binding agents, such as antibodies, that specifically bind a tumor-associated antigen (TAA). These binding agents may be referred to as “TAA-binding agents”. In some embodiments, the TAA-binding agent (e.g., an antibody) comprises an exogenous polypeptide comprising at least one antigenic peptide. In some embodiments, the antigenic peptide comprises a T-cell epitope. In some embodiments, T-cell epitope is a CD8+ T-cell epitope. In some embodiments, the T-cell epitope is a MHC Class I-restricted epitope. In some embodiments, the T-cell epitope is a MHC Class II-restricted epitope. In some embodiments, the exogenous polypeptide is derived from a virus. In some embodiments, the exogenous polypeptide is derived from a virus that includes but is not limited to measles virus, varicella-zoster virus, influenza virus, mumps virus, poliovirus, rubella virus, rotavirus, hepatitis A virus (HAV), hepatitis B virus (HBV), and cytomegalovirus (CMV).

In some embodiments, the TAA-binding agent is an antibody that comprises an exogenous polypeptide comprising at least one antigenic peptide, wherein the exogenous polypeptide is part of the light chain of the antibody. In some embodiments, the light chain of the antibody comprises the exogenous polypeptide. In some embodiments, the exogenous polypeptide is attached to the N-terminus of the light chain of the antibody. In some embodiments, the exogenous polypeptide is attached to the C-terminus of the light chain of the antibody. In some embodiments, the exogenous polypeptide is within the variable region of the light chain of the antibody. In some embodiments, the exogenous polypeptide is within a CDR of the light chain of the antibody. In some embodiments, the exogenous polypeptide is near CDR1 of the light chain of the antibody. In some embodiments, the exogenous polypeptide is embedded within CDR1 of the light chain of the antibody. In some embodiments, the exogenous polypeptide is part of CDR1 of the light chain of the antibody. In some embodiments, the exogenous polypeptide is near CDR2 of the light chain of the antibody. In some embodiments, the exogenous polypeptide is embedded within CDR2 of the light chain of the antibody. In some embodiments, the exogenous polypeptide is part of CDR2 of the light chain of the antibody. In some embodiments, the exogenous polypeptide is near CDR3 of the light chain of the antibody. In some embodiments, the exogenous polypeptide is embedded within CDR3 of the light chain of the antibody. In some embodiments, the exogenous polypeptide is part of CDR3 of the light chain of the antibody. In some embodiments, the exogenous polypeptide is part of the framework region(s) of the light chain of the antibody. In some embodiments, the exogenous polypeptide is part of the framework region preceding CDR1 of the light chain of the antibody. In some embodiments, the exogenous polypeptide is part of the framework region between CDR1 and CDR2 of the light chain of the antibody. In some embodiments, the exogenous polypeptide is part of the framework region between CDR2 and CDR3 of the light chain of the antibody. In some embodiments, the exogenous polypeptide is part of the framework region following CDR3 of the light chain of the antibody. In some embodiments, the exogenous polypeptide is part of a constant region of the light chain of the antibody. In some embodiments, the exogenous polypeptide replaces part of a constant region of the light chain of the antibody.

In some embodiments, the TAA-binding antigen is an antibody that comprises an exogenous polypeptide comprising at least one antigenic peptide, wherein the exogenous polypeptide is part of the heavy chain of the antibody. In some embodiments, the heavy chain of the antibody comprises the exogenous polypeptide. In some embodiments, the exogenous polypeptide is attached to the N-terminus of the heavy chain of the antibody. In some embodiments, the exogenous polypeptide is attached to the C-terminus of the heavy chain of the antibody. In some embodiments, the exogenous polypeptide is within the variable region of the heavy chain of the antibody. In some embodiments, the exogenous polypeptide is within a CDR of the heavy chain of the antibody. In some embodiments, the exogenous polypeptide is near CDR1 of the heavy chain of the antibody. In some embodiments, the exogenous polypeptide is embedded within CDR1 of the heavy chain of the antibody. In some embodiments, the exogenous polypeptide is part of CDR1 of the heavy chain of the antibody. In some embodiments, the exogenous polypeptide is near CDR2 of the heavy chain of the antibody. In some embodiments, the exogenous polypeptide is embedded within CDR2 of the heavy chain of the antibody. In some embodiments, the exogenous polypeptide is part of CDR2 of the heavy chain of the antibody. In some embodiments, the exogenous polypeptide is near CDR3 of the heavy chain of the antibody. In some embodiments, the exogenous polypeptide is embedded within CDR3 of the 1 heavy chain of the antibody. In some embodiments, the exogenous polypeptide is part of CDR3 of the heavy chain of the antibody. In some embodiments, the exogenous polypeptide is part of the framework region(s) of the heavy chain of the antibody. In some embodiments, the exogenous polypeptide is part of the framework region preceding CDR1 of the heavy chain of the antibody. In some embodiments, the exogenous polypeptide is part of the framework region between CDR1 and CDR2 of the heavy chain of the antibody. In some embodiments, the exogenous polypeptide is part of the framework region between CDR2 and CDR3 of the heavy chain of the antibody. In some embodiments, the exogenous polypeptide is part of the framework region following CDR3 of the heavy chain of the antibody. In some embodiments, the exogenous polypeptide is part of the hinge region of the heavy chain of the antibody. In some embodiments, the exogenous polypeptide is embedded within the hinge region of the heavy chain of the antibody. In some embodiments, the exogenous polypeptide is part of a constant region of the heavy chain of the antibody. In some embodiments, the exogenous polypeptide replaces part of a constant region of the heavy chain of the antibody.

In some embodiments, the TAA-binding antigen is an antibody that comprises an exogenous polypeptide comprising at least one antigenic peptide, wherein the antibody is a single chain antibody. As used herein, a “single chain antibody” is an antibody wherein the heavy chain of the antibody is linked to the light chain of the antibody. In some embodiments, the antibody is a single chain antibody wherein the heavy chain and the light chain are linked by an amino acid sequence. In some embodiments, the amino acid sequence between the heavy chain and the light chain comprises the exogenous polypeptide.

In some embodiments, the TAA-binding agent is an antibody that specifically binds a tumor-associated antigen or TAA. As used herein, “TAA” comprises proteins that are expressed on tumor cells at a higher level than on normal cells. The term “TAA” also comprises proteins that have mutations resulting in altered proteins or “neoantigens” that generally are not found on normal tissues or cells. The term “TAA” also comprises fragments of the proteins, such as the extracellular domain or a fragment of the extracellular domain. In some embodiments, the TAA-binding agent is an antibody that specifically binds a TAA that includes, but is not limited to, B7-H4/VTCN1, B7-H3, PVRL4, DLL3, CD20, CEA, MUC16, FOLH1, SLC45A3, OXTR, CDH3, GABRP, ECEL1, SIGLEC11, and DIO3. In some embodiments, the TAA-binding agent comprises an antibody that specifically binds a TAA that includes, but is not limited to, B7-H4/VTCN1, B7-H3, PVRL4, DLL3, CD20, CEA, MUC16, FOLH1, SLC45A3, OXTR, CDH3, GABRP, ECEL1, SIGLEC11, and DIO3. In some embodiments, a TAA includes, but is not limited to, HER2, CD47, mucin 1 (MUC-1), carcinoembryonic antigen (CEA), telomerase reverse transcriptase (TERT), and Wilms' tumor gene (WT1). In some embodiments, the TAA is selected from the group consisting of ACPP, STEAP1, STEAP2, GPA33, GUCY2C, PSA, PSMA, PAP, PSCA, TARP, and GARP. In some embodiments, the TAA-binding agent is an antibody that specifically binds a TAA that includes, but is not limited to, STEAP2 (six-transmembrane epithelial antigen of the prostate 2), STEAP1 (six-transmembrane epithelial antigen of the prostate 1), PSA (prostate-specific antigen), PSMA (prostate-specific membrane antigen), PAP (prostatic acid phosphatase), PSCA (prostate stem cell antigen), and TARP (T-cell receptor gamma alternate reading frame protein). In some embodiments, the TAA-binding agent is an antibody that specifically binds B7-H4. In some embodiments, the TAA-binding agent comprises an antibody that specifically binds B7-H4. In some embodiments, the TAA-binding agent is an antibody that specifically binds GABRP. In some embodiments, the TAA-binding agent comprises an antibody that specifically binds GABRP. In some embodiments, the TAA-binding agent is an antibody that specifically binds CDH3 (cadherin 3; p-cadherin). In some embodiments, the TAA-binding agent comprises an antibody that specifically binds CDH3.

In some embodiments, the TAA-binding agent is a monoclonal antibody. In some embodiments, the TAA-binding agent is a humanized antibody. In some embodiments, the TAA-binding agent is a human antibody. In some embodiments, the TAA-binding agent is a recombinant antibody or a chimeric antibody. In some embodiments, the TAA-binding agent a bispecific antibody. In some embodiments, the TAA-binding agent is an antibody fragment comprising an antigen binding site. In some embodiments, the TAA-binding agent is an IgG antibody. In some embodiments, the TAA-binding agent is an IgG1 antibody, an IgG2 antibody, or an IgG4 antibody.

In some embodiments, the TAA-binding agent is an antibody that specifically binds B7-H4. In some embodiments, the TAA-binding agent comprises an antibody that specifically binds B7-H4. In some embodiments, the TAA-binding agent comprises an antibody that specifically binds B7-H4, wherein the antibody (e.g., a humanized antibody) comprises: (a) a heavy chain CDR1 comprising TSYYMH (SEQ ID NO:9), a heavy chain CDR2 comprising YVDPFNGGTSYNQKFKG (SEQ ID NO:10), and a heavy chain CDR3 comprising FIAGFAN (SEQ ID NO:11) or IAGFAN (SEQ ID NO:12) and/or (b) a light chain CDR1 comprising KASQDIKSYLS (SEQ ID NO:13), a light chain CDR2 comprising YATSLAD (SEQ ID NO:14), and a light chain CDR3 comprising LQHGESPYT (SEQ ID NO:15) or LQHGESPY (SEQ ID NO:16).

In some embodiments of the invention, the TAA-binding agent is an antibody that delivers an exogenous polypeptide comprising at least one antigenic peptide to a tumor cell. In some embodiments, the antibody specifically binds a TAA and delivers the exogenous polypeptide comprising at least one antigenic peptide to a tumor cell expressing the TAA. In some embodiments, the antibody binds the TAA and is internalized by the tumor cell. In some embodiments, the antibody binds the TAA, is internalized by the tumor cell, and the antibody with the exogenous polypeptide is processed by the cell. In some embodiments, the antibody binds the TAA, is internalized by the tumor cell, and the antigenic peptide is presented on the surface of the tumor cell. In some embodiments, the antibody binds the TAA, is internalized by the tumor cell, the antibody with the exogenous polypeptide is processed by the cell, and the antigenic peptide is presented on the surface of the tumor cell.

In some embodiments of the invention, the TAA-binding agent is an antibody that delivers an exogenous polypeptide comprising at least one antigenic peptide to a tumor cell, wherein the antigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments of the invention, the TAA-binding agent is an antibody that delivers an exogenous polypeptide comprising at least one antigenic peptide to a tumor cell, wherein the antigenic peptide is presented on the surface of the tumor cell, and an immune response against the tumor cell is induced. In some embodiments, the immune response against the tumor cell is enhanced. In some embodiments, the immune response against the tumor cell is increased. In some embodiments, the TAA-binding agent is an antibody that delivers an exogenous polypeptide comprising at least one antigenic peptide to a tumor cell, wherein the antigenic peptide is presented on the surface of the tumor cell, and tumor growth is inhibited.

In some embodiments of the invention, the TAA-binding agent is an antibody that delivers an exogenous polypeptide comprising at least one antigenic peptide to a tumor cell, wherein the antigenic peptide is presented on the surface of the tumor cell, and T-cell killing directed against the tumor cell is induced. In some embodiments, T-cell killing directed against the tumor cell is enhanced. In some embodiments, T-cell killing directed against the tumor cell is increased.

In certain embodiments of each of the aforementioned aspects, as well as other aspects and/or embodiments described elsewhere herein, the TAA-binding agent is isolated. In certain embodiments of each of the aforementioned aspects, as well as other aspects and/or embodiments described elsewhere herein, the TAA-binding agent is substantially pure.

The invention further provides cells that comprise the TAA-binding agents (e.g., antibodies) described herein. The invention also provides cells that produce the TAA-binding agents (e.g., antibodies) described herein. In some embodiments, the cell is a prokaryotic cell. In some embodiments, the cell is an eukaryotic cell. In some embodiments, the cell is a mammalian cell.

In another aspect, the invention provides isolated polynucleotide molecules comprising a polynucleotide that encodes the TAA-binding agents and/or polypeptides of each of the aforementioned aspects, as well as other aspects and/or embodiments described herein.

The invention further provides expression vectors that comprise the polynucleotides, as well as cells that comprise the expression vectors and/or the polynucleotides. In some embodiments, the cell is a prokaryotic cell. In some embodiments, the cell is an eukaryotic cell.

Compositions comprising a TAA-binding agent (e.g., an antibody and/or polypeptide) described herein are provided. Pharmaceutical compositions comprising a TAA-binding agent (e.g., an antibody and/or polypeptide) described herein and a pharmaceutically acceptable carrier are further provided.

In another aspect, the invention provides methods of using the TAA-binding agents (e.g., antibodies and/or polypeptides) described herein. In some embodiments, a method of inhibiting growth of a tumor comprises contacting the tumor with an effective amount of a TAA-binding agent described herein. In some embodiments, a method of inhibiting growth of a tumor in a subject comprises administering to the subject a therapeutically effective amount of a TAA-binding agent described herein. In some embodiments, a method of increasing the immunogenicity of a tumor comprises contacting the tumor with an effective amount of a TAA-binding agent described herein. In some embodiments, a method of increasing the immunogenicity of a tumor in a subject comprises administering to the subject a therapeutically effective amount of a TAA-binding agent described herein. In some embodiments, a method of reducing the tumorigenicity of a tumor in a subject comprises administering to the subject a therapeutically effective amount of a TAA-binding agent described herein. In some embodiments, a method of reducing the tumorigenicity of a tumor in a subject by reducing the frequency of cancer stem cells in the tumor comprises administering to the subject a therapeutically effective amount of a TAA-binding agent described herein. In some embodiments, a method of reducing the frequency of cancer stem cells in a tumor in a subject comprises administering to the subject a therapeutically effective amount of a TAA-binding agent described herein. In some embodiments, the tumor is selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor. In some embodiments, the tumor is a solid tumor.

In some embodiments of the methods of the invention, a method comprises treating cancer. In some embodiments, a method of treating cancer in a subject, comprises administering to the subject a therapeutically effective amount of a TAA-binding agent described herein. In some embodiments, the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer. In some embodiments, the cancer is a solid cancer.

In some embodiments of the methods of the invention, a method comprises redirecting an existing immune response to a tumor. In some embodiments, a method of redirecting an existing immune response to a tumor, comprises administering to a subject a therapeutically effective amount of a TAA-binding agent described herein. In some embodiments, the existing immune response is to a virus. In some embodiments, the existing immune response is a cell-mediated response. In some embodiments, the existing immune response comprises cytotoxic T-cells.

In some embodiments of the methods described herein, a method comprises administering at least one additional therapeutic agent. In some embodiments, the additional therapeutic agent is a chemotherapeutic agent. In some embodiments, the additional therapeutic agent is an antibody. In some embodiments, the additional therapeutic agent is an immunotherapeutic agent. In some embodiments, the immunotherapeutic agent is selected from the group consisting of: GM-CSF, M-CSF, G-CSF, IL-2, IL-3, IL-12, IL-15, B7-1 (CD80), B7-2 (CD86), 4-1BB ligand, GITR ligand (GITRL), OX-40 ligand (OX-40L), CD40 ligand (CD40L), anti-CD3 antibody, anti-CTLA-4 antibody, anti-PD-1 antibody, anti-PD-L1 antibody, anti-GITR antibody, anti-OX-40 antibody, anti-CD40 antibody, anti-4-1BB antibody, anti-LAG-3 antibody, and anti-TIM-3 antibody. In some embodiments, the additional therapeutic agent is an inhibitor of the Notch pathway, the Wnt pathway, or the RSPO/LGR pathway.

In some embodiments of the methods described herein, the subject has had a tumor or a cancer removed. In some embodiments of the methods described herein, the subject has had a tumor or a cancer previously treated.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIG. 1. Schematic depictions of a representative number of the antibodies proposed that contain an exogenous polypeptide comprising at least one antigenic peptide.

FIG. 2. Antigen presentation by tumor cells. KP_LUN31 lung tumor cells were incubated with soluble ovalbumin (250 μg/ml or 500 μg/ml) in the presence or absence of the proteasome inhibitor lactacystin. Cells were co-cultured with B3Z T-cells and T-cell activation was evaluated by determination of IL-2 production.

FIG. 3. Antigen presentation by tumor cells. KP_LUN31 lung tumor cells were incubated with soluble ovalbumin (OVA at 250 μg/ml or 500 μg/ml) in the presence or absence of the scavenger receptor inhibitor polyinosinic acid (Poly(I)). Cells were co-cultured with B3Z T-cells and T-cell activation was evaluated by determination of IL-2 production.

FIG. 4. Antigen presentation after targeted antibody binding. B16F10, KP_LUN31, RSPO2-expressing B16F10, or RSPO-expressing KP_LUN31 tumor cells were incubated with anti-RSPO2 antibody 130M23 or anti-RSPO2 antibody 130M23-SIINFEKL. Cells were co-cultured with B3Z T-cells and T-cell activation was evaluated by determination of IL-2 production.

FIG. 5. Cytotoxicity assay. B16F10 or RSPO2-expressing B16F10 cells were incubated with anti-RSPO2 antibody 130M23 or anti-RSPO2 antibody 130M23-SIINFEKL and then labeled with calcein for use as targets. Splenocytes were isolated from OT-I transgenic mice and stimulated with the OVA peptide SIINFEKL to generate effector T-cells. Targets and effector cells were co-cultured and specific killing was calculated from the release of the calcein.

FIG. 6. Antigen presentation after targeted antibody binding. MC38 or RSPO2-expressing MC38 tumor cells were incubated with anti-RSPO2 antibody 130M23 or anti-RSPO2 antibody 130M23-SIINFEKL. Cells were co-cultured with B3Z T-cells and T-cell activation was evaluated by determination of IL-2 production.

FIG. 7. Antigen presentation after targeted antibody binding. TC-1 or RSPO2-expressing TC-1 tumor cells were incubated with anti-RSPO2 antibody 130M23 or anti-RSPO2 antibody 130M23-SIINFEKL. Cells were co-cultured with B3Z T-cells and T-cell activation was evaluated by determination of IL-2 production.

FIG. 8. FACS analysis of RSPO2 expression of RSPO2-expressing MC38 cell clones. RSPO2-expressing MC38 cell clones were incubated with anti-RSPO2 antibody 130M23 or anti-RSPO2 antibody 130M23-SIINFEKL. Cells were co-cultured with B3Z T-cells and T-cell activation was evaluated by determination of IL-2 production.

FIG. 9. RSPO2 expression levels and MHC class I antigen expression levels on RSPO2-expressing B16F10, KP_LUN31, MC38, and TC-1 tumor cells.

FIG. 10. Antigen presentation after targeted antibody binding. B7-H4-expressing MC38 tumor cells or the parental cell line were incubated with anti-mB7-H4 antibody 278M6 or anti-mB7-H7 antibody 278M6-SIINFEKL. Cells were co-cultured with B3Z T-cells and T-cell activation was evaluated by determination of IL-2 production.

FIG. 11. Antigen presentation after targeted antibody binding. B7-H4-expressing TC-1 tumor cells or the parental cell line were incubated with anti-mB7-H4 antibody 278M6 or anti-mB7-H7 antibody 278M6-SIINFEKL. Cells were co-cultured with B3Z T-cells and T-cell activation was evaluated by determination of IL-2 production.

FIG. 12. FACS analysis of OT-I activated T-cells.

FIG. 13. Tumor growth of E.G7-OVA or MC38-OVA tumor cells after adoptive transfer of OT-I activated T-cells.

FIG. 14. Tumor growth of RSPO2-expressing MC38 tumor cells after treatment with anti-RSPO2 antibody 130M23-SIINFEKL in the absence or presence of OT-I activated T-cells.

### DETAILED DESCRIPTION OF THE INVENTION

The present invention provides novel agents, including, but not limited to, antibodies that modulate the immune response. The agents include antibodies that specifically bind tumor-associated antigens and redirect an existing immune response to target a tumor cell.

An immune response is generally divided into innate and adaptive immunity. Innate immunity occurs immediately, when circulating innate cells recognize a problem. Adaptive immunity occurs later, as it relies on the coordination and expansion of specific adaptive immune cells. Immune memory or immunological memory follows the adaptive response, when mature adaptive cells, highly specific to the original pathogen, are retained for later use.

Adaptive immune cells are specialized, with each B-cell or T-cell bearing unique receptors, B-cell receptors (BCRs) and T-cell receptors (TCRs). Each receptor recognizes a specific antigen, which is simply any molecule that may bind to a BCR or TCR. Antigens are derived from a variety of sources including pathogens (e.g., viruses and bacteria), host cells, and allergens. Antigens are typically processed by particular immune cells and presented to B-cells and/or T-cells.

A critical determinant of T-cell activation is recognition by the TCR of a T-cell of an antigen complexed with a MHC molecule on the surface of a “presenting” cell. This highly tuned mechanism allows T-cells to survey cells throughout the body for the presence of foreign antigens. For example, this mechanism allows T-cells to recognize cells that are infected with a virus or cells that express a protein that has acquired a mutation. Mutation is cellular proteins can result in presentation of a novel peptide sequence or “neoantigen” by a MHC molecule. While a number of immunotherapeutic agents have achieved dramatic efficacy in some patients with tumors, it is clear that not all patients derive benefit from these immunotherapeutic agents and some tumor types appear to be resistant. Tumors with high somatic mutation prevalence appear to have a greater frequency of neoantigens that make these cells more immunogenic and better targets for a strong immune response. However, there are many tumors that appear to have low levels of somatic mutations and neoantigens and therefore may have a low immunogenic capability that leads to the lack of a potent anti-tumor immune response (Schumacher and Schreiber, 2015, Science, 348:69-74).

Most subjects have “immunological memory” to a variety of common pathogens. Immunological memory is the ability of the immune system to respond more rapidly and effectively to foreign agents/pathogens that have been encountered previously. This response reflects the preexistence of a clonally expanded population of antigen-specific lymphocytes. Subjects can have a primary exposure to a pathogen, for example a virus, by a natural infection or by vaccination, but whether through infection or vaccination, subjects exposed to the pathogen develop long-term protection against the pathogen. This state of protection is a consequence of immunological memory. This immunological memory can be harnessed and redirected to attack other targets. This may be referred to as “redirected immune response” or “redirected immunotherapy”.

Thus this invention is based on redirecting an existing immune response that normally targets cells expressing foreign antigens such as viral antigens, to target unwanted cells such as tumor cells or cancer cells. In some embodiments, the existing immune response (or immunological memory) is due to a subject having encountered a pathogen, such as a virus, through a natural infection. In some embodiments, the existing immune response is due to a subject having been vaccinated against a specific pathogen. The presence of immunological memory against a specific pathogen within any given population will depend upon numerous factors, including but not limited to, the age of the subject, where the subject lives, the prevalence of vaccination within the population and/or country, the economic level of the subject and/or population. In the U.S., it is common to be vaccinated against measles virus, varicella-zoster virus (VZV; chicken pox), influenza, mumps virus, rotavirus, poliovirus, rubella virus, hepatitis A virus and hepatitis B virus. In addition, most people have been naturally infected with cytomegalovirus (CMV) and Epstein Barr virus (EBV). Therefore, in some embodiments, the invention comprises methods of harnessing the immunological memory against a viral antigen (e.g., existing immune response due to a vaccination) and redirect cytolytic T-cells to a new target, such as a tumor cell expressing the viral antigen.

In some embodiments, a method comprises delivering to tumor cells in a subject an antigen derived from a known pathogen, the antigen is processed and presented by MHC protein on the surface of the tumor cells, and the subject's existing immunological memory against the known antigen is redirected to target the tumor. A particular antigenic peptide can be embedded within a protein that selectively targets the tumor, such as an antibody with specificity towards a tumor-associated antigen (TAA). The agent (e.g., an antibody) upon binding to the TAA is internalized, is processed and degraded, and peptides derived from the agent, including the embedded antigen peptide, are presented on the cell surface of the tumor cell in complex with a MHC molecule. The antigenic peptide complexed with a MHC molecule presented on the surface of the tumor cell is recognized by an appropriate T-cell receptor that is able to activate a cytotoxic T-cell response directed to the tumor.

### I. Definitions

To facilitate an understanding of the present invention, a number of terms and phrases are defined below.

The terms “modulation” and “modulate” as used herein refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a protein, a pathway, a system, or other biological targets of interest.

The term “antibody” as used herein refers to an immunoglobulin molecule that recognizes and specifically binds a target through at least one antigen-binding site. The target may be a protein, polypeptide, peptide, carbohydrate, polynucleotide, lipid, or a combination of any of the foregoing. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and generally refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of an antibody light chain or the variable region of an antibody heavy chain, either alone or in combination. Generally, the variable region of a heavy chain or a light chain consists of four framework regions connected by three complementarity determining regions (CDRs), also known as “hypervariable regions”. The CDRs in each chain are held together in close proximity by the framework regions and, with the CDRs from the other chain, contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies that recognize different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody fragment, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which amino acid residues of the CDRs are replaced by amino acid residues from the CDRs of a non-human species (e.g., mouse, rat, rabbit, or hamster) that have the desired specificity, affinity, and/or binding capability. In some instances, the framework variable region amino acid residues of a human immunoglobulin may be replaced with the corresponding amino acid residues in an antibody from a non-human species. The humanized antibody can be further modified by the substitution of additional amino acid residues either in the framework variable region and/or within the replaced non-human amino acid residues to further refine and optimize antibody specificity, affinity, and/or binding capability. The humanized antibody may comprise variable domains containing all or substantially all of the CDRs that correspond to the non-human immunoglobulin, whereas all or substantially all of the framework variable regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable regions of the light and heavy chains correspond to the variable regions of an antibody derived from one species of mammal (e.g., mouse, rat, rabbit, etc.) with the desired specificity, affinity, and/or binding capability, while the constant regions are homologous to the sequence in an antibody derived from another species. The constant regions are usually human to avoid eliciting an immune response in the antibody.

The term “T-cell epitope” as used herein refers to a sequence of amino acids or a peptide that can be recognized by a T-cell receptor. T-cell epitopes presented by MHC class I molecules are typically peptides between 8 and 11 amino acids in length. T-cell epitopes presented by MHC class II molecules are typically longer peptides, ranging from 13-17 amino acids in length.

The terms “selectively binds” or “specifically binds” mean that an agent interacts more frequently, more rapidly, with greater duration, with greater affinity, or with some combination of the above to the epitope, protein, or target molecule than with alternative substances, including related and unrelated proteins. In certain embodiments “specifically binds” means, for instance, that an agent binds a protein or target with a K_{D} of about 0.1 mM or less, but more usually less than about 1 μM. In certain embodiments, “specifically binds” means that an agent binds a target with a K_{D} of at least about 0.1 μM or less, at least about 0.01 μM or less, or at least about 1 nM or less. Because of the sequence identity between homologous proteins in different species, specific binding can include an agent that recognizes a protein or target in more than one species. Likewise, because of homology within certain regions of polypeptide sequences of different proteins, specific binding can include an agent that recognizes more than one protein or target. It is understood that, in certain embodiments, an agent that specifically binds a first target may or may not specifically bind a second target. As such, “specific binding” does not necessarily require (although it can include) exclusive binding, i.e. binding to a single target. Thus, an agent may, in certain embodiments, specifically bind more than one target. In certain embodiments, multiple targets may be bound by the same antigen-binding site on the agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because some of the polypeptides of this invention may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, the coding region of a nucleotide sequence.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies of the invention do not abrogate the binding of the polypeptide or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes cell-mediated and/or humoral immune responses. It includes, but is not limited to, both T-cell and B-cell responses, as well as responses from other cells of the immune system such as natural killer (NK) cells, monocytes, macrophages, etc.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. Generally, a “metastatic” or “metastasizing” cell is one that loses adhesive contacts with neighboring cells and migrates via the bloodstream or lymph from the primary site of disease to secondary sites throughout the body.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an appropriate host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into appropriate hosts (e.g., mice).

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an agent described herein, an antibody, a polypeptide, a polynucleotide, a small organic molecule, or other drug effective to “treat” a disease or disorder in a subject such as, a mammal. In the case of cancer or a tumor, the therapeutically effective amount of an agent (e.g., an antibody) has a therapeutic effect and as such can enhance or boost the immune response, enhance or boost the anti-tumor response, increase cytolytic activity of immune cells, increase killing of tumor cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Antibodies to Tumor-Associated Antigens

In general, a basic principle of tumor immunology is that cancer cells express antigens that differentiate them from non-cancer cells. However, many tumor-associated antigens (TAA) are not necessarily tumor-specific and may also be found on normal tissues. These antigens are often products of mutated cellular genes, aberrantly expressed normal genes, or genes encoding viral proteins. TAAs may include, but are not limited to, differentiation antigens, mutational antigens, over-expressed cellular antigens, viral antigens, and cancer antigens that are expressed in germ cells of the testis and ovary but are silent in normal somatic cells. In some embodiments, a TAA is expressed at high levels in cancer tissues as compared to the level of expression normal tissues. In some embodiments, a TAA is over-expressed in cancer tissues but not expressed or not highly expressed in normal tissues. In some embodiments, a TAA is over-expressed in cancer tissues but not expressed in normal tissues except for in dispensable tissues (e.g., prostate, breast, ovary). In some embodiments, a TAA is expressed at the cell surface.

TAAs can be identified by a number of methods known to those of skill in the art. In some embodiments, a TAA is identified from a database, for example, the data compiled at the Cancer Immunity journal (cancerimmunity.org) comprising tumor antigens including tumor antigens resulting from mutations, shared tumors-specific antigens, differentiation antigens, and antigens over-expressed in tumors. In some embodiments, a genome-wide cDNA microarray analysis is used. This technology provides information of gene expression profiles in normal and tumor/cancer tissues.

Particular examples of TAAs include, but are not limited to, those described in the TANTIGEN database available from Bioinformatics Core at Cancer Vaccine Center, Dana-Farber Cancer Institute. Non-limiting examples include: 707AP, ANKRD30A, DCT, GPR143, KLK3, KLK4, MC1R, MLANA, OCA2, RAB38, SCGB2A2, SILV, SOX2, TYR, TYRP1, XAGE1, ABCC3, ACPP, ADAM17, ADFP, AFP, AIM2, ALDH1A1, ALK, AML1, ART4, BCL-2, BCL2L1, BIRC5, BIRC7, BST2, CA9, CCNI, CCNB1, CCND1, CEL, CEACAM5, CLCA2, CPSF1, CSPG4, CSF1, CYP1B1, DDR1, DEK, DKK1, EGFR, ENAH, EPHA2, EPHA3, ERBB2, ETV5, EZH2, FGF5, F4.2, FMNL1, FOLH1, GPC3, HSPA1A, IL13RA2, KAAG1, MCL1, MDM2, MMP2, MRPL28, MSLN, MUC1, MUC2, NPM1, PAX3, PPIB, PRAME, RAGE, RGS5, RHAMM, RNF43, SART1, SART3, SCRN1, SFMBT1, SOX10, SOX11, SOX4. STEAP1, SYND1, TACSTD1, TERT, TOP2A, TOP2B, TP53, TPBG, TRG, TRIM68, TRPM8, TSPYL1, WDR46, WT1, XBP1, ZNF395, ANXA2, BATE, CCDC110, CDAG2, CTAG1, CTAG2, CXORF61, GAGE1, GAGE2, GAGE3, GAGE4, GAGE5, GAGE6, GAGE7, HERV-K-MEL, GAGE8, MAGEA1, MAGEA10, MAGEA12, MAGEA2, MAGEA3, MAGEA4, MAGEA6, MAGEA9, MAGEB1, MAGEB2, MAGEC2, MGAT5, SAGE1, SPA17, SSX2, SSX4, SYCP1, TGFBR2, VENTXP1, ABI2, ABL1, ACRBP, AKAP13, APC ARTC1, ATIC, BAAT, BCAP31, BCR, BTBD2, CALR3, CAN, CDC2, CDKN1A, COTL1, CTSH, DNAJC8, EIF4EBP1, ETV6, FMOD, FOXO1, FUT1, H3F3A, HSMD, HMHA1, HMOX1, HSPE, HNRPL, IER3, IGF2BP3, ITGB8, ITPR2, JUP, LCK, LDLR, LGALS3BP, LRP1, LY6K, MAGED4, MET, MGFE8, MFI2, MMP14, OAS3, PA2G4, PAGE4, PAK2, PARP12, PGK1, PML1, PRTN3, PSCA, PTHLH, PXDNL, RARA, RCVRN, RPA1, RPL10A, RPS2, RPSA, SDCBP, SEPT2, SLPB, SLC35A4, SLC45A3, SSX1, STAT1, SUPT7L, SYT, TAPBP, TOR3A, TPM4, TRGC2, TTK, TYMS, UBE2A, UBE2V1, WHSC2, and WNK2.

In some embodiments, a TAA is selected from the group consisting of: B7-H4/VTCN1, B7-H3, PVRL4, DLL3, CD20, CEA, MUC16, FOLH1, SLC45A3, OXTR, CDH3, GABRP, ECEL1, SIGLEC11, and DIO3. In some embodiments, a TAA is selected from the group consisting of: HER2, CD47, mucin 1 (MUC-1), carcinoembryonic antigen (CEA), telomerase reverse transcriptase (TERT), and Wilms' tumor gene (WT1). In some embodiments, a TAA is selected from the group consisting of: STEAP2 (six-transmembrane epithelial antigen of the prostate 2), STEAP1 (six-transmembrane epithelial antigen of the prostate 1), PSA (prostate-specific antigen), PSMA (prostate-specific membrane antigen), PAP (prostatic acid phosphatase), PSCA (prostate stem cell antigen), and TARP (T-cell receptor gamma alternate reading frame protein). In some embodiments, the TAA is selected from the group consisting of ACPP, GPA33, GUCY2C, and GARP. In some embodiments, the TAA is GABRP. In some embodiments, the TAA is GARP. In some embodiments, the TAA is STEAP1. In some embodiments, the TAA is STEAP2. In some embodiments, the TAA is CDH3.

In some embodiments, the TAA is B7-H4 (also known as V-set domain-containing T-cell activation inhibitor 1 or VTCN1). B7-H4 is a type I transmembrane protein that belongs to the B7 superfamily B7-H4 and has been shown to play a role in the negative regulation of T-cell immunity. B7-H4 is widely expressed in tumor tissues including breast cancer, cervical cancer, bladder cancer, lung cancer, renal cell carcinoma, prostate cancer, ovarian cancer, and pancreatic cancer. The full-length amino acid (aa) sequence of human B7-H4 (UniProtKB No. Q7Z7D3) is known in the art and is provided herein as SEQ ID NO:6. As used herein, reference to amino acid positions refer to the numbering of the full-length amino acid sequence including the signal sequence.

TAAs can be targeted by specific binding agents to deliver antigenic peptides to a tumor cell. In some embodiments, the antigenic peptide is presented on the surface of the tumor cell and increases the immunogenicity of the tumor. In some embodiments, a TAA-binding agent is an antibody that specifically binds a specific TAA.

In certain embodiments, a TAA-binding agent (e.g., an antibody) binds a particular TAA with a dissociation constant (K_{D}) of about 1 μM or less, about 100 nM or less, about 40 nM or less, about 20 nM or less, about 10 nM or less, about 1 nM or less, or about 0.1 nM or less. In certain embodiments, a TAA-binding agent binds a particular TAA with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a TAA-binding agent binds a particular TAA with a K_{D} of about 20 nM or less. In some embodiments, a TAA-binding agent binds TAA with a K_{D} of about 10 nM or less. In some embodiments, a TAA-binding agent binds a particular TAA with a K_{D} of about 1 nM or less. In some embodiments, a TAA-binding agent binds a particular TAA with a K_{D} of about 0.5 nM or less. In some embodiments, a TAA-binding agent binds a particular TAA with a K_{D} of about 0.1 nM or less. In some embodiments, the dissociation constant of the binding agent (e.g., an antibody) to a particular TAA is the dissociation constant determined using a TAA fusion protein comprising at least a portion of the extracellular domain of TAA immobilized on a Biacore chip. In some embodiments, the dissociation constant of the binding agent (e.g., an antibody) to TAA is the dissociation constant determined using the binding agent captured by an anti-human IgG antibody on a Biacore chip and a soluble TAA protein.

In certain embodiments, the TAA-binding agent (e.g., an antibody) binds a particular TAA with a half maximal effective concentration (EC₅₀) of about 104 or less, about 100 nM or less, about 40 nM or less, about 20 nM or less, about 10 nM or less, about 1 nM or less, or about 0.1 nM or less. In certain embodiments, a TAA-binding agent binds a particular TAA with a half maximal effective concentration (EC₅₀) of about 104 or less, about 100 nM or less, about 40 nM or less, about 20 nM or less, about 10 nM or less, about 1 nM or less, or about 0.1 nM or less.

In certain embodiments, the TAA-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In certain embodiments, the antibody is an IgG1 antibody. In certain embodiments, the antibody is an IgG2 antibody. In some embodiments, the antibody is an IgG4 antibody. In some embodiments, the antibody is a fusion protein. In some embodiments, the antibody is a single chain antibody. In some embodiments, the antibody comprises a linker between the light chain and the heavy chain. In certain embodiments, the antibody is an antibody fragment comprising an antigen-binding site. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is conjugated to a cytotoxic moiety. In some embodiments, the antibody is isolated. In some embodiments, the antibody is substantially pure.

In some embodiments, the TAA-binding agents are polyclonal antibodies. Polyclonal antibodies can be prepared by any known method. In some embodiments, polyclonal antibodies are produced by immunizing an animal (e.g., a rabbit, rat, mouse, goat, donkey) with an antigen of interest (e.g., a purified peptide fragment, full-length recombinant protein, or fusion protein) using multiple subcutaneous or intraperitoneal injections. The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from the immunized animal, usually from blood or ascites. The polyclonal antibodies can be purified from serum or ascites according to standard methods in the art including, but not limited to, affinity chromatography, ion-exchange chromatography, gel electrophoresis, and dialysis.

In some embodiments, a TAA-binding agent is a monoclonal antibody. Monoclonal antibodies can be prepared using hybridoma methods known to one of skill in the art. In some embodiments, using the hybridoma method, a mouse, rat, rabbit, hamster, or other appropriate host animal, is immunized as described above to elicit the production of antibodies that specifically bind the immunizing antigen. In some embodiments, lymphocytes can be immunized in vitro. In some embodiments, the immunizing antigen can be a human protein or a fragment thereof. In some embodiments, the immunizing antigen can be a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the nucleotides encoding the heavy and light chain variable regions of the antibody or the entire heavy and light chains, and their sequence is determined using standard techniques. The isolated polynucleotides encoding the heavy and light chain variable regions or heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species.

The polynucleotide(s) encoding a monoclonal antibody can be modified, for example, by using recombinant DNA technology to generate alternative antibodies. In some embodiments, the constant domains of the light chain and heavy chain of, for example, a mouse monoclonal antibody can be substituted for constant regions of, for example, a human antibody to generate a chimeric antibody, or for a non-immunoglobulin polypeptide to generate a fusion antibody. In some embodiments, the constant regions are truncated or removed to generate a desired antibody fragment of a monoclonal antibody. Site-directed or high-density mutagenesis of the variable region(s) can be used to optimize specificity, affinity, etc. of a monoclonal antibody.

In some embodiments, a TAA-binding agent is a humanized antibody. Typically, humanized antibodies are human immunoglobulins in which the amino acid residues of the CDRs are replaced by amino acid residues from CDRs of a non-human species (e.g., mouse, rat, rabbit, hamster, etc.) that have the desired specificity, affinity, and/or binding capability using methods known to one skilled in the art. In some embodiments, some of the framework variable region amino acid residues of a human immunoglobulin are replaced with corresponding amino acid residues in an antibody from a non-human species. In some embodiments, a humanized antibody can be further modified by the substitution of additional residues either in the framework variable region and/or within the replaced non-human residues to further refine and optimize antibody specificity, affinity, and/or capability. In general, a humanized antibody will comprise variable regions containing all, or substantially all, of the CDRs that correspond to the non-human immunoglobulin whereas all, or substantially all, of the framework regions are those of a human immunoglobulin sequence. In some embodiments, the framework regions are those of a human consensus immunoglobulin sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject.

In certain embodiments, a TAA-binding agent is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated. In some embodiments, the human antibody can be selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production.

In some embodiments, the TAA-binding agent is a bispecific antibody. Thus, this invention encompasses bispecific antibodies that specifically recognize a particular TAA and at least one additional target. Bispecific antibodies are capable of specifically recognizing and binding at least two different antigens or epitopes. The different epitopes can either be within the same molecule (e.g., two epitopes on a TAA) or on different molecules (e.g., one epitope on a TAA and one epitope on a different protein). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., a tumor and/or tumor microenvironment). In some embodiments, a bispecific antibody has the ability to concentrate the actions of two agents to a common target (e.g., a tumor or a tumor cell). In some embodiments, a bispecific antibody has the ability to target the actions of two agents to more than one biological pathway or function.

In some embodiments, the bispecific antibody is a monoclonal antibody. In some embodiments, the bispecific antibody is a humanized antibody. In some embodiments, the bispecific antibody is a human antibody. In some embodiments, the bispecific antibody is an IgG1 antibody. In some embodiments, the bispecific antibody is an IgG2 antibody. In some embodiments, the bispecific antibody is an IgG4 antibody. In some embodiments, the bispecific antibody has decreased toxicity and/or side effects. In some embodiments, the bispecific antibody has decreased toxicity and/or side effects as compared to a mixture of the two individual antibodies or the antibodies as single agents. In some embodiments, the bispecific antibody has an increased therapeutic index. In some embodiments, the bispecific antibody has an increased therapeutic index as compared to a mixture of the two individual antibodies or the antibodies as single agents.

In some embodiments, the antibodies can be used to direct cytotoxic agents to cells which express a particular TAA. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art. In some embodiments, the bispecific antibodies comprise heavy chain constant regions with modifications in the amino acids which are part of the interface between the two heavy chains. In some embodiments, the bispecific antibodies can be generated using a “knobs-into-holes” strategy. In some cases, the “knobs” and “holes” terminology is replaced with the terms “protuberances” and “cavities”. In some embodiments, the bispecific antibodies may comprise variant hinge regions incapable of forming disulfide linkages between the heavy chains. In some embodiments, the modifications may comprise changes in amino acids that result in altered electrostatic interactions. In some embodiments, the modifications may comprise changes in amino acids that result in altered hydrophobic/hydrophilic interactions.

Bispecific antibodies can be intact antibodies or antibody fragments comprising antigen-binding sites. Antibodies with more than two valencies are also contemplated. For example, trispecific antibodies can be prepared. Thus, in certain embodiments the antibodies to a particular TAA are multispecific.

In certain embodiments, the antibodies (or other polypeptides) described herein may be monospecific. In certain embodiments, each of the one or more antigen-binding sites that an antibody contains is capable of binding (or binds) a homologous epitope on a particular TAA.

In certain embodiments, a TAA-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced by recombinant methods. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for TAAs or derivatives, fragments, analogs or homologs thereof. In some embodiments, antibody fragments are linear antibody fragments. In certain embodiments, antibody fragments are monospecific or bispecific. In certain embodiments, the TAA-binding agent is a scFv. Various techniques can be used for the production of single-chain antibodies specific to a particular TAA.

In some embodiments, especially in the case of antibody fragments, an antibody is modified in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the particular TAA. In this regard, the variable region may comprise or be derived from any type of mammal that can be induced to mount a humoral response and generate immunoglobulins against the desired antigen. As such, the variable region of the modified antibodies can be, for example, of human, murine, rat, rabbit, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen binding capacity of one variable domain to another. Rather, it may only be necessary to transfer those residues that are required to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain-deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding. Similarly, it may be desirable to simply delete the part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or providing for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the modified antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase the serum half-life of the antibody. In other embodiments, the constant region modifications reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques.

In certain embodiments, a TAA-binding agent that is an antibody does not have one or more effector functions. For instance, in some embodiments, the antibody has no ADCC activity, and/or no complement-dependent cytotoxicity (CDC) activity. In certain embodiments, the antibody does not bind an Fc receptor, and/or complement factors. In certain embodiments, the antibody has no effector function(s).

The present invention further embraces variants and equivalents which are substantially homologous to the recombinant, monoclonal, chimeric, humanized, and human antibodies, or antibody fragments thereof, described herein. These variants can contain, for example, conservative substitution mutations, i.e. the substitution of one or more amino acids by similar amino acids.

The present invention provides methods for producing an antibody that binds a particular TAA, including bispecific antibodies that specifically bind both a particular TAA and a second target. In some embodiments, the method for producing an antibody that binds a particular TAA comprises using hybridoma techniques. In some embodiments, a method for producing an antibody that binds a particular human TAA is provided. In some embodiments, the method comprises using a polypeptide comprising the extracellular domain of a human TAA or a fragment thereof as an antigen. In some embodiments, the method of generating an antibody that binds a particular TAA comprises screening a human phage library. The present invention further provides methods of identifying an antibody that binds a particular TAA. In some embodiments, the antibody is identified by FACS screening for binding to a TAA or a fragment thereof. In some embodiments, the antibody is identified by screening using ELISA for binding to a TAA or a fragment thereof. In some embodiments, the antibody is identified by screening by FACS for blocking of binding of a TAA to a known receptor or ligand.

In some embodiments, a method of generating an antibody to a TAA comprises immunizing a mammal with a polypeptide comprising the extracellular domain of the TAA. In some embodiments, a method of generating an antibody to a TAA comprises immunizing a mammal with a polypeptide comprising a fragment of the extracellular domain of the TAA. In some embodiments, the method further comprises isolating antibodies or antibody-producing cells from the mammal. In some embodiments, a method of generating a monoclonal antibody which binds a TAA comprises: (a) immunizing a mammal with a polypeptide comprising the extracellular domain or a fragment thereof of a TAA; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds the TAA. In certain embodiments, the mammal is a mouse. In some embodiments, the mammal is a rat. In some embodiments, the mammal is a rabbit.

In some embodiments, a method of producing an antibody that binds a particular TAA comprises identifying an antibody using a membrane-bound heterodimeric molecule comprising a single antigen-binding site. In some non-limiting embodiments, the antibody is identified using methods and polypeptides described in International Publication WO 2011/100566.

In some embodiments, a method of producing an antibody that binds a particular TAA comprises screening an antibody-expressing library. In some embodiments, the antibody-expressing library is a phage library. In some embodiments, the screening comprises panning. In some embodiments, the antibody-expressing library is a mammalian cell library. In some embodiments, the antibody-expressing library is screened using the extracellular domain or a fragment thereof of the TAA.

In certain embodiments, the antibodies described herein are isolated. In certain embodiments, the antibodies described herein are substantially pure.

The TAA-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

In a non-limiting example, screening for specific binding of an antibody to a particular human TAA may be determined using ELISA. An ELISA comprises preparing antigen (e.g., the TAA or a fragment thereof), coating wells of a 96-well microtiter plate with antigen, adding the test antibodies conjugated to a detectable compound such as an enzymatic substrate (e.g. horseradish peroxidase or alkaline phosphatase) to the well, incubating for a period of time and detecting the presence of an antibody bound to the antigen. In some embodiments, the test antibodies are not conjugated to a detectable compound, but instead a secondary antibody that recognizes the antibody (e.g., an anti-Fc antibody) and is conjugated to a detectable compound is added to the wells. In some embodiments, instead of coating the well with the antigen, the test antibodies can be coated to the wells, the antigen (e.g., TAA) is added to the wells, followed by a secondary antibody conjugated to a detectable compound. One of skill in the art would be knowledgeable as to the parameters that can be modified to increase the signal detected as well as other variations of ELISAs known in the art.

In another non-limiting example, the specific binding of an antibody to a particular TAA may be determined using FACS. A FACS screening assay may comprise generating a cDNA construct that expresses an antigen as a full-length protein (TAA) or a fusion protein (e.g., TAA ECD-CD4TM), transfecting the construct into cells, expressing the antigen on the surface of the cells, mixing the test antibodies with the transfected cells, and incubating for a period of time. The cells bound by the test antibodies may be identified using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

In some embodiments, a TAA-binding agent comprises an antibody that specifically binds B7-H4. In some embodiments, the antibody specifically binds mouse B7-H4. In some embodiments, the antibody specifically binds human B7-H4. In some embodiments, the antibody specifically binds mouse B7-H4 and human B7-H4. In some embodiments, the TAA-binding agent comprises an antibody that specifically binds B7-H4, wherein the antibody comprises a heavy chain CDR1 comprising TSYYMH (SEQ ID NO:9), a heavy chain CDR2 comprising YVDPFNGGTSYNQKFKG (SEQ ID NO:10), and a heavy chain CDR3 comprising FIAGFAN (SEQ ID NO:11) or IAGFAN (SEQ ID NO:12). In some embodiments, the antibody further comprises a light chain CDR1 comprising KASQDIKSYLS (SEQ ID NO:13), a light chain CDR2 comprising YATSLAD (SEQ ID NO:14), and a light chain CDR3 comprising LQHGESPYT (SEQ ID NO:15) or LQHGESPY (SEQ ID NO:16). In some embodiments, the TAA-binding agent comprises an antibody that specifically binds B7-H4, wherein the antibody comprises a light chain CDR1 comprising KASQDIKSYLS (SEQ ID NO:13), a light chain CDR2 comprising YATSLAD (SEQ ID NO:14), and a light chain CDR3 comprising LQHGESPYT (SEQ ID NO:15) or LQHGESPY (SEQ ID NO:16). In some embodiments, the TAA-binding agent comprises an antibody that specifically binds B7-H4, wherein the antibody comprises: (a) a heavy chain CDR1 comprising TSYYMH (SEQ ID NO:9), a heavy chain CDR2 comprising YVDPFNGGTSYNQKFKG (SEQ ID NO:10), and a heavy chain CDR3 comprising FIAGFAN (SEQ ID NO:11) or IAGFAN (SEQ ID NO:12) and (b) a light chain CDR1 comprising KASQDIKSYLS (SEQ ID NO:13), a light chain CDR2 comprising YATSLAD (SEQ ID NO:14), and a light chain CDR3 comprising LQHGESPYT (SEQ ID NO:15) or LQHGESPY (SEQ ID NO:16).

In certain embodiments, the TAA-binding agent comprises an antibody that specifically binds B7-H4, wherein the antibody comprises: (a) a heavy chain CDR1 comprising TSYYMH (SEQ ID NO:9), or a variant thereof comprising 1, 2, 3, or 4 amino acid substitutions; (b) a heavy chain CDR2 comprising YVDPFNGGTSYNQKFKG (SEQ ID NO:10), or a variant thereof comprising 1, 2, 3, or 4 amino acid substitutions; (c) a heavy chain CDR3 comprising FIAGFAN (SEQ ID NO:11), IAGFAN (SEQ ID NO:12), or a variant thereof comprising 1, 2, 3, or 4 amino acid substitutions; (d) a light chain CDR1 comprising KASQDIKSYLS (SEQ ID NO:13), or a variant thereof comprising 1, 2, 3, or 4 amino acid substitutions; (e) a light chain CDR2 comprising YATSLAD (SEQ ID NO:114), or a variant thereof comprising 1, 2, 3, or 4 amino acid substitutions; and (f) a light chain CDR3 comprising LQHGESPYT (SEQ ID NO:15), LQHGESPY (SEQ ID NO:16), or a variant thereof comprising 1, 2, 3, or 4 amino acid substitutions. In certain embodiments, the amino acid substitutions are conservative substitutions. In some embodiments, the substitutions are made as part of a humanization process. In some embodiments, the substitutions are made as part of a germline humanization process.

In certain embodiments, the TAA-binding agent comprises an antibody that specifically binds B7-H4, wherein the antibody comprises a heavy chain variable region having at least about 80% sequence identity to SEQ ID NO:17 and/or a light chain variable region having at least 80% sequence identity to SEQ ID NO:18. In certain embodiments, the antibody comprises a heavy chain variable region having at least about 85%, at least about 90%, at least about 95%, at least about 97%, or at least about 99% sequence identity to SEQ ID NO:17. In certain embodiments, the antibody comprises a light chain variable region having at least about 85%, at least about 90%, at least about 95%, at least about 97%, or at least about 99% sequence identity to SEQ ID NO:18. In certain embodiments, the antibody comprises a heavy chain variable region having at least about 95% sequence identity to SEQ ID NO:17 and/or a light chain variable region having at least about 95% sequence identity to SEQ ID NO:18. In certain embodiments, the antibody comprises a heavy chain variable region comprising SEQ ID NO:17 and/or a light chain variable region comprising SEQ ID NO:18. In certain embodiments, the antibody comprises a heavy chain variable region comprising SEQ ID NO:17 and a light chain variable region comprising SEQ ID NO:18.

In certain embodiments, the TAA-binding agent comprises an antibody that specifically binds B7-H4, wherein the antibody comprises: (a) a heavy chain having at least 90% sequence identity to SEQ ID NO:20 and/or (b) a light chain having at least 90% sequence identity to SEQ ID NO:22. In some embodiments, the antibody comprises: (a) a heavy chain having at least 95% sequence identity to SEQ ID NO:20 and/or (b) a light chain having at least 95% sequence identity to SEQ ID NO:22. In some embodiments, the antibody comprises a heavy chain comprising SEQ ID NO:20 and/or a light chain comprising SEQ ID NO:22. In some embodiments, the antibody comprises a heavy chain comprising SEQ ID NO:20 and a light chain comprising SEQ ID NO:22.

In certain embodiments, a TAA-binding agent comprises an antibody that specifically binds B7-H4, wherein the antibody comprises the heavy chain CDRs and light chain CDRs of antibody 278M1. In some embodiments, the antibody comprises the heavy chain variable region and light chain variable region of antibody 278M1. In some embodiments, the antibody comprises the variable regions of antibody 278M1 wherein the heavy chain variable region and/or the light chain variable region from the 278M1 antibody have been affinity-matured. In certain embodiments, the antibody comprises the heavy chain and light chain of antibody 278M1 (with or without the leader sequence). In certain embodiments, a TAA-binding agent comprises antibody 278M1. In certain embodiments, a TAA-binding agent comprises an antibody that specifically binds B7-H4, wherein the antibody comprises the heavy chain variable region and/or the light chain variable region of antibody 278M1 wherein the heavy chain variable region and/or the light chain variable region have been humanized. In certain embodiments, the antibody comprises the heavy chain variable region and/or the light chain variable region of antibody 278M1 in a humanized form. In certain embodiments, the antibody comprises the heavy chain variable region of antibody 278M1 as part of an IgG1, IgG2, or IgG4 heavy chain.

The binding affinity of an antibody or other binding agent to an antigen (e.g., a TAA) and the off-rate of an antibody-antigen interaction can be determined by competitive binding assays. One example of a competitive binding assay is a radioimmunoassay comprising the incubation of labeled antigen (e.g., ³H or ¹²⁵I-TAA), or fragment or variant thereof, with the antibody of interest in the presence of increasing amounts of unlabeled antigen followed by the detection of the antibody bound to the labeled antigen. The affinity of the antibody for the antigen and the binding off-rates can be determined from the data by Scatchard plot analysis. In some embodiments, Biacore kinetic analysis is used to determine the binding on and off rates of antibodies or agents that bind an antigen (e.g., a TAA). In some embodiments, Biacore kinetic analysis comprises analyzing the binding and dissociation of antibodies from chips with immobilized antigen (e.g., a TAA) on their surface. In some embodiments, Biacore kinetic analysis comprises analyzing the binding and dissociation of antigen (e.g., a TAA) from chips with immobilized antibody (e.g., anti-TAA antibody) on their surface.

The TAA-binding agents (e.g., antibodies) of the present invention can be further modified to comprise an exogenous polypeptide. In some embodiments, the TAA-binding agent that comprises an exogenous polypeptide is a fusion protein. In some embodiments, TAA-binding agent is not conjugated to the exogenous polypeptide. In some embodiments, an exogenous polypeptide comprises at least one antigenic peptide. In some embodiments, the antigenic peptide comprises a T-cell epitope. In some embodiments, T-cell epitope is a CD8+ T-cell epitope (also referred to as a CTL epitope). In some embodiments, the T-cell epitope is a MHC Class I-restricted epitope. In some embodiments, the T-cell epitope is a MHC Class II-restricted epitope. In some embodiments, the exogenous polypeptide is derived from a virus. In some embodiments, the exogenous polypeptide is derived from a virus that includes but is not limited to measles virus, varicella-zoster virus (VZV; chicken pox virus), influenza virus, mumps virus, poliovirus, rubella virus, rotavirus, hepatitis A virus (HAV), hepatitis B virus (HBV), Epstein Barr virus (EBV), and cytomegalovirus (CMV). As discussed herein and is known by those of skill in the art, CD8+ T-cell epitopes originate from proteins that are cleaved into short peptides by the proteasome, are processed, and are non-covalently associated with MHC Class I molecules. These epitopes range from 8-11 amino acids, although a majority of them have been observed to be nine-mers. An epitope associates with a MHC Class I molecule only if its affinity to the MHC molecule is sufficiently high. In some embodiments, the T-cell epitope is derived from a virus that includes but is not limited to measles virus, varicella-zoster virus (VZV; chicken pox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the T-cell epitope is derived from measles virus. In some embodiments, the T-cell epitope is derived from cytomegalovirus. In some embodiments, the T-cell epitope is derived from varicella-zoster virus.

In humans, MHC molecules are encoded by the Human Leukocyte Antigen (HLA) locus, which is the most polymorphic locus in the human genome. There are 3 major MHC class I genes in the HLA locus—HLA-A, HLA-B, HLA-C and 3 minor genes—HLA-E, HLA-F and HLA-G. Within the HLA-A, HLA-B, and HLA-C genes there are thousands of different alleles and within each population there is a different distribution of allele frequencies. Individuals that display different sets of alleles with potentially different binding specificities (HLA-restriction) are likely to react to a different set of antigenic peptides from any given pathogen. There are some antigenic peptides that bind to many HLA Class I molecules and are recognized by more the one T-cell receptor. These antigenic peptides are considered to be “promiscuous epitopes”. There are some HLA molecules that share overlapping peptide repertories and those are considered “supertypes”.

Antigenic peptides, e.g., CD8+ T-cell epitopes, for use in the TAA-binding agents described herein, can be identified by a variety of methods. Peptide mapping can be undertaken in predictive and experimental systems. Algorithms have been developed to test the potential capacity of a given peptide to bind a predetermined MHC allele. In addition, databases have been created that aggregate biomedical information in regard to epitope information from publications that describe immune epitopes. One database is the Immune Epitope Database and Analysis Resource (IEDB; iedb.org) which was created in 2003 and is continually being updated (see, Salimi et al., 2012, Immunology, 137:117-123). The IEDB database includes epitope prediction tools for peptides and MHC Class I molecules as well as peptides and MHC Class II molecules and B-cell epitopes. Some databases have tools for T-cell epitope prediction based on MHC binding, as well as tools for prediction of antigenic peptide fragments produced upon processing by the proteasome. Some publicly available MHC Class I prediction tools include, but are not limited to, BIMAS, RANKPEP, SYFPEITHI, NetMHC, IEDB, TEPITOPE, MULTIPRED2, and MultiRTA. Some publically available T-cell epitope prediction tools include, but are not limited to, EpicCapo, POPISK, EpiMatrix, NetCTLpan, iMatrix, IEDB tool, and CTLPred.

Experimental systems may include, for example, T-cell activation and/or ELISPOT assays that are well-known in the art. In a non-limiting example, a number of identified T-cell peptides are evaluated in ELISPOT assays. A standard ELISPOT is used to measure antigen-specific effector T-cells. PBMCs from donors with known HLA types are added with or without peptide to an ELISPOT plate coated with an antibody specific for the cytokine of interest (e.g., IFN-gamma) and incubated overnight. The cytokine released by the cells binds to the capture antibody and detected by a second antibody and cells producing the cytokine are identified by a substrate that produces colored spots. A culture ELISPOT assay is used to measure antigen-specific memory T-cells. Isolated PBMCs are cultured with or without peptide for 10 days with the addition of IL-2 at day 3 and day 7 allowing the expansion of antigen-specific T-cells. The cells are harvested, washed, and restimulated within a standard ELISPOT assay. Generally, spots are counted using an automated ELISPOT reader. Results can be shown as the relative fold change of spots with peptide compared to spots without peptide. (See, for example, Calarota et al., 2013, Clinical and Developmental Immunology, Article ID 637649).

In some embodiments, the TAA-binding agent is an antibody that comprises an exogenous polypeptide comprising at least one antigenic peptide, wherein the exogenous polypeptide is part of the light chain of the antibody. In some embodiments, the light chain of the antibody comprises the exogenous polypeptide. In some embodiments, the exogenous polypeptide is attached to the N-terminus of the light chain of the antibody. In some embodiments, the exogenous polypeptide is attached to the C-terminus of the light chain of the antibody. In some embodiments, the exogenous polypeptide is within the variable region of the light chain of the antibody. In some embodiments, the exogenous polypeptide is within a CDR of the light chain of the antibody. In some embodiments, the exogenous polypeptide is near CDR1 of the light chain of the antibody. In some embodiments, the exogenous polypeptide is embedded within CDR1 of the light chain of the antibody. In some embodiments, the exogenous polypeptide is part of CDR1 of the light chain of the antibody. In some embodiments, the exogenous polypeptide is near CDR2 of the light chain of the antibody. In some embodiments, the exogenous polypeptide is embedded within CDR2 of the light chain of the antibody. In some embodiments, the exogenous polypeptide is part of CDR2 of the light chain of the antibody. In some embodiments, the exogenous polypeptide is near CDR3 of the light chain of the antibody. In some embodiments, the exogenous polypeptide is embedded within CDR3 of the light chain of the antibody. In some embodiments, the exogenous polypeptide is part of CDR3 of the light chain of the antibody. In some embodiments, the exogenous polypeptide is part of the framework region(s) of the light chain of the antibody. In some embodiments, the exogenous polypeptide is part of the framework region preceding CDR1 of the light chain of the antibody. In some embodiments, the exogenous polypeptide is part of the framework region between CDR1 and CDR2 of the light chain of the antibody. In some embodiments, the exogenous polypeptide is part of the framework region between CDR2 and CDR3 of the light chain of the antibody. In some embodiments, the exogenous polypeptide is part of the framework region following CDR3 of the light chain of the antibody. In some embodiments, the exogenous polypeptide is part of a constant region of the light chain of the antibody. In some embodiments, the exogenous polypeptide replaces part of a constant region of the light chain of the antibody. In some embodiments, the TAA-binding agent is an antibody that comprises an exogenous polypeptide comprising at least one antigenic peptide, wherein the exogenous polypeptide and the light chain of the antibody are a fusion protein.

In some embodiments, the light chain of the antibody comprises at least one antigenic peptide (e.g., a T-cell epitope). In some embodiments, the at least one antigenic peptide (e.g., a T-cell epitope) is attached to the N-terminus of the light chain of the antibody. In some embodiments, the at least one antigenic peptide (e.g., a T-cell epitope) is attached to the C-terminus of the light chain of the antibody. In some embodiments, the at least one antigenic peptide (e.g., a T-cell epitope) is within the variable region of the light chain of the antibody. In some embodiments, the at least one antigenic peptide (e.g., a T-cell epitope) is within a CDR of the light chain of the antibody. In some embodiments, the at least one antigenic peptide (e.g., a T-cell epitope) is near CDR1 of the light chain of the antibody. In some embodiments, the at least one antigenic peptide (e.g., a T-cell epitope) is embedded within CDR1 of the light chain of the antibody. In some embodiments, the at least one antigenic peptide (e.g., a T-cell epitope) is part of CDR1 of the light chain of the antibody. In some embodiments, the at least one antigenic peptide (e.g., a T-cell epitope) is near CDR2 of the light chain of the antibody. In some embodiments, the at least one antigenic peptide (e.g., a T-cell epitope) is embedded within CDR2 of the light chain of the antibody. In some embodiments, the at least one antigenic peptide (e.g., a T-cell epitope) is part of CDR2 of the light chain of the antibody. In some embodiments, the at least one antigenic peptide (e.g., a T-cell epitope) is near CDR3 of the light chain of the antibody. In some embodiments, the at least one antigenic peptide (e.g., a T-cell epitope) is embedded within CDR3 of the light chain of the antibody. In some embodiments, the at least one antigenic peptide (e.g., a T-cell epitope) is part of CDR3 of the light chain of the antibody. In some embodiments, the at least one antigenic peptide (e.g., a T-cell epitope) is part of the framework region(s) of the light chain of the antibody. In some embodiments, the at least one antigenic peptide (e.g., a T-cell epitope) is part of the framework region preceding CDR1 of the light chain of the antibody. In some embodiments, the at least one antigenic peptide (e.g., a T-cell epitope) is part of the framework region between CDR1 and CDR2 of the light chain of the antibody. In some embodiments, the at least one antigenic peptide (e.g., a T-cell epitope) is part of the framework region between CDR2 and CDR3 of the light chain of the antibody. In some embodiments, the at least one antigenic peptide (e.g., a T-cell epitope) is part of the framework region following CDR3 of the light chain of the antibody. In some embodiments, the exogenous polypeptide (e.g., a T-cell epitope) is part of a constant region of the light chain of the antibody. In some embodiments, the exogenous polypeptide (e.g., a T-cell epitope) replaces part of a constant region of the light chain of the antibody. In some embodiments, the TAA-binding agent is an antibody comprising at least one antigenic peptide, wherein the antigenic peptide(s) and the light chain of the antibody are a fusion protein.

In some embodiments, the TAA-binding antigen is an antibody that comprises an exogenous polypeptide comprising at least one antigenic peptide, wherein the exogenous polypeptide is part of the heavy chain of the antibody. In some embodiments, the heavy chain of the antibody comprises the exogenous polypeptide. In some embodiments, the exogenous polypeptide is attached to the N-terminus of the heavy chain of the antibody. In some embodiments, the exogenous polypeptide is attached to the C-terminus of the heavy chain of the antibody. In some embodiments, the exogenous polypeptide is within the variable region of the heavy chain of the antibody. In some embodiments, the exogenous polypeptide is within a CDR of the heavy chain of the antibody. In some embodiments, the exogenous polypeptide is near CDR1 of the heavy chain of the antibody. In some embodiments, the exogenous polypeptide is embedded within CDR1 of the heavy chain of the antibody. In some embodiments, the exogenous polypeptide is part of CDR1 of the heavy chain of the antibody. In some embodiments, the exogenous polypeptide is near CDR2 of the heavy chain of the antibody. In some embodiments, the exogenous polypeptide is embedded within CDR2 of the heavy chain of the antibody. In some embodiments, the exogenous polypeptide is part of CDR2 of the heavy chain of the antibody. In some embodiments, the exogenous polypeptide is near CDR3 of the heavy chain of the antibody. In some embodiments, the exogenous polypeptide is embedded within CDR3 of the 1 heavy chain of the antibody. In some embodiments, the exogenous polypeptide is part of CDR3 of the heavy chain of the antibody. In some embodiments, the exogenous polypeptide is part of the framework region(s) of the heavy chain of the antibody. In some embodiments, the exogenous polypeptide is part of the framework region preceding CDR1 of the heavy chain of the antibody. In some embodiments, the exogenous polypeptide is part of the framework region between CDR1 and CDR2 of the heavy chain of the antibody. In some embodiments, the exogenous polypeptide is part of the framework region between CDR2 and CDR3 of the heavy chain of the antibody. In some embodiments, the exogenous polypeptide is part of the framework region following CDR3 of the heavy chain of the antibody. In some embodiments, the exogenous polypeptide is part of the hinge region of the heavy chain of the antibody. In some embodiments, the exogenous polypeptide is embedded within the hinge region of the heavy chain of the antibody. In some embodiments, the exogenous polypeptide is part of a constant region of the heavy chain of the antibody. In some embodiments, the exogenous polypeptide replaces part of a constant region of the heavy chain of the antibody. In some embodiments, the TAA-binding agent is an antibody that comprises an exogenous polypeptide comprising at least one antigenic peptide, wherein the exogenous polypeptide and the heavy chain of the antibody are a fusion protein.

In some embodiments, the heavy chain of the antibody comprises at least one antigenic peptide (e.g., a T-cell epitope). In some embodiments, the at least one antigenic peptide (e.g., a T-cell epitope) is attached to the N-terminus of the heavy chain of the antibody. In some embodiments, the at least one antigenic peptide (e.g., a T-cell epitope) is attached to the C-terminus of the heavy chain of the antibody. In some embodiments, the at least one antigenic peptide (e.g., a T-cell epitope) is within the variable region of the heavy chain of the antibody. In some embodiments, the at least one antigenic peptide (e.g., a T-cell epitope) is within a CDR of the heavy chain of the antibody. In some embodiments, the at least one antigenic peptide (e.g., a T-cell epitope) is near CDR1 of the heavy chain of the antibody. In some embodiments, the at least one antigenic peptide (e.g., a T-cell epitope) is embedded within CDR1 of the heavy chain of the antibody. In some embodiments, the at least one antigenic peptide (e.g., a T-cell epitope) is part of CDR1 of the heavy chain of the antibody. In some embodiments, the at least one antigenic peptide (e.g., a T-cell epitope) is near CDR2 of the heavy chain of the antibody. In some embodiments, the at least one antigenic peptide (e.g., a T-cell epitope) is embedded within CDR2 of the heavy chain of the antibody. In some embodiments, the at least one antigenic peptide (e.g., a T-cell epitope) is part of CDR2 of the heavy chain of the antibody. In some embodiments, the at least one antigenic peptide (e.g., a T-cell epitope) is near CDR3 of the heavy chain of the antibody. In some embodiments, the at least one antigenic peptide (e.g., a T-cell epitope) is embedded within CDR3 of the heavy chain of the antibody. In some embodiments, the at least one antigenic peptide (e.g., a T-cell epitope) is part of CDR3 of the heavy chain of the antibody. In some embodiments, the at least one antigenic peptide (e.g., a T-cell epitope) is part of the framework region(s) of the heavy chain of the antibody. In some embodiments, the at least one antigenic peptide (e.g., a T-cell epitope) is part of the framework region preceding CDR1 of the heavy chain of the antibody. In some embodiments, the at least one antigenic peptide (e.g., a T-cell epitope) is part of the framework region between CDR1 and CDR2 of the heavy chain of the antibody. In some embodiments, the at least one antigenic peptide (e.g., a T-cell epitope) is part of the framework region between CDR2 and CDR3 of the heavy chain of the antibody. In some embodiments, the at least one antigenic peptide (e.g., a T-cell epitope) is part of the framework region following CDR3 of the heavy chain of the antibody. In some embodiments, the at least one antigenic peptide (e.g., a T-cell epitope) is part of the hinge region of the heavy chain of the antibody. In some embodiments, the at least one antigenic peptide (e.g., a T-cell epitope) is embedded within the hinge region of the heavy chain of the antibody. In some embodiments, the at least one antigenic peptide (e.g., a T-cell epitope) is part of a constant region of the heavy chain of the antibody. In some embodiments, the at least one antigenic peptide (e.g., a T-cell epitope) replaces part of a constant region of the heavy chain of the antibody. In some embodiments, the TAA-binding agent is an antibody comprising at least one antigenic peptide, wherein the antigenic peptide(s) and the heavy chain of the antibody are a fusion protein.

In some embodiments, the TAA-binding antigen is an antibody that comprises an exogenous polypeptide comprising at least one antigenic peptide, wherein the antibody is a single chain antibody. As used herein, a “single chain antibody” is an antibody wherein the heavy chain of the antibody is linked to the light chain of the antibody. In some embodiments, the antibody is a single chain antibody wherein the heavy chain and the light chain are linked by an amino acid sequence. In some embodiments, the amino acid sequence between the heavy chain and the light chain comprises the exogenous polypeptide. In some embodiments, the amino acid sequence between the heavy chain and the light chain comprises at least one antigenic peptide (e.g., a T-cell epitope). In some embodiments, the antibody is a fusion protein comprising the heavy chain of the antibody, the light chain of the antibody, and the exogenous polypeptide. In some embodiments, the antibody is a fusion protein comprising the heavy chain of the antibody, the light chain of the antibody, and the at least one antigenic peptide.

In some embodiments, a TAA-binding agent comprises a fusion protein comprising (i) an antibody that specifically binds a TAA and (ii) at least one antigenic peptide. In some embodiments, the fusion protein comprises (i) an antibody that specifically binds a TAA and (ii) an exogenous polypeptide comprising at least one T-cell epitope. In some embodiments, the fusion protein comprises (i) an antibody that specifically binds a TAA and (ii) an exogenous polypeptide comprising at least one MHC Class I-restricted T-cell epitope. In some embodiments, a TAA-binding agent comprises a fusion protein comprising (i) an antibody that specifically binds a TAA and (ii) at least one antigenic peptide, wherein (a) the fusion protein is internalized by a tumor cell after binding to the TAA; (b) the antigenic peptide is processed and presented on the surface of the tumor cell associated with a MHC class I molecule; and (c) the peptide/MHC Class I complex is recognized by cytotoxic T-cells.

This invention also encompasses homodimeric agents/molecules and heterodimeric agents/molecules comprising a TAA-binding agent described herein. In some embodiments, the homodimeric agents are polypeptides. In some embodiments, the heterodimeric molecules are polypeptides. Generally, the homodimeric molecule comprises two identical polypeptides. Generally, the heterodimeric molecule comprises at least two different polypeptides. In some embodiments, the heterodimeric molecule is capable of binding at least two targets, e.g., a bispecific agent. The targets may be, for example, two different proteins on a single cell or two different proteins on two separate cells. The term “arm” may be used herein to describe the structure of a homodimeric molecule, a heterodimeric molecule, and/or a bispecific agent. In some embodiments, each arm comprises at least one polypeptide. Generally, each arm of a heterodimeric molecule has a different function, for example, binding two different targets. In some embodiments, one arm may comprise an antigen-binding site from an antibody. In some embodiments, one arm may comprise a binding portion of a receptor. In some embodiments, a homodimeric agent comprises two identical arms. In some embodiments, a heterodimeric agent comprises two different arms. In some embodiments, a bispecific agent comprises two different arms.

In some embodiments, a TAA-binding agent is a homodimeric molecule. In some embodiments, the homodimeric molecule comprises two identical polypeptides. In some embodiments, a TAA-binding agent is a heterodimeric molecule. In some embodiments, the heterodimeric molecule comprises at least two different polypeptides. In some embodiments, a TAA-binding agent is a heterodimeric agent. In some embodiments, a TAA-binding agent is a bispecific agent. In certain embodiments, the TAA-binding agent is a bispecific antibody.

In some embodiments, the TAA-binding agent is a heterodimeric molecule (e.g., a bispecific agent) that comprises a first CH3 domain and a second CH3 domain, each of which is modified to promote formation of heteromultimers. In some embodiments, the first and second CH3 domains are modified using a knobs-into-holes technique. In some embodiments, the first and second CH3 domains comprise changes in amino acids that result in altered electrostatic interactions. In some embodiments, the first and second CH3 domains comprise changes in amino acids that result in altered hydrophobic/hydrophilic interactions.

In some embodiments, the TAA-binding agent is a bispecific agent that comprises heavy chain constant regions selected from the group consisting of: (a) a first human IgG1 constant region, wherein the amino acids corresponding to positions 253 and 292 of IgG1 (SEQ ID NO:1) are replaced with glutamate or aspartate, and a second human IgG1 constant region, wherein the amino acids corresponding to positions 240 and 282 of IgG1 (SEQ ID NO:1) are replaced with lysine; (b) a first human IgG2 constant region, wherein the amino acids corresponding to positions 249 and 288 of IgG2 (SEQ ID NO:2) are replaced with glutamate or aspartate, and a second human IgG2 constant region wherein the amino acids corresponding to positions 236 and 278 of IgG2 (SEQ ID NO:2) are replaced with lysine; (c) a first human IgG3 constant region, wherein the amino acids corresponding to positions 300 and 339 of IgG3 (SEQ ID NO:3) are replaced with glutamate or aspartate, and a second human IgG3 constant region wherein the amino acids corresponding to positions 287 and 329 of IgG3 (SEQ ID NO:3) are replaced with lysine; and (d) a first human IgG4 constant region, wherein the amino acids corresponding to positions 250 and 289 of IgG4 (SEQ ID NO:4) are replaced with glutamate or aspartate, and a second IgG4 constant region wherein the amino acids corresponding to positions 237 and 279 of IgG4 (SEQ ID NO:4) are replaced with lysine.

In some embodiments, the TAA-binding agent is a bispecific agent which comprises a first human IgG1 constant region with amino acid substitutions at positions corresponding to positions 253 and 292 of IgG1 (SEQ ID NO:1), wherein the amino acids at positions corresponding to positions 253 and 292 of IgG1 (SEQ ID NO:1) are replaced with glutamate or aspartate, and a second human IgG1 constant region with amino acid substitutions at positions corresponding to positions 240 and 282 of IgG1 (SEQ ID NO:1), wherein the amino acids at positions corresponding to positions 240 and 282 of IgG1 (SEQ ID NO:1) are replaced with lysine. In some embodiments, the TAA-binding agent is a bispecific antibody which comprises a first human IgG2 constant region with amino acid substitutions at positions corresponding to positions 249 and 288 of IgG2 (SEQ ID NO:2), wherein the amino acids at positions corresponding to positions 249 and 288 of IgG2 (SEQ ID NO:2) are replaced with glutamate or aspartate, and a second human IgG2 constant region with amino acid substitutions at positions corresponding to positions 236 and 278 of IgG2 (SEQ ID NO:2), wherein the amino acids at positions corresponding to positions 236 and 278 of IgG2 (SEQ ID NO:2) are replaced with lysine. In some embodiments, the TAA-binding agent is a bispecific antibody which comprises a first human IgG3 constant region with amino acid substitutions at positions corresponding to positions 300 and 339 of IgG3 (SEQ ID NO:3), wherein the amino acids at positions corresponding to positions 300 and 339 of IgG3 (SEQ ID NO:3) are replaced with glutamate or aspartate, and a second human IgG3 constant region with amino acid substitutions at positions corresponding to positions 287 and 329 of IgG3 (SEQ ID NO:3), wherein the amino acids at positions corresponding to positions 287 and 329 of IgG3 (SEQ ID NO:3) are replaced with lysine. In some embodiments, the TAA-binding agent is a bispecific antibody which comprises a first human IgG4 constant region with amino acid substitutions at positions corresponding to positions 250 and 289 of IgG4 (SEQ ID NO:4), wherein the amino acids at positions corresponding to positions 250 and 289 of IgG4 (SEQ ID NO:4) are replaced with glutamate or aspartate, and a second human IgG4 constant region with amino acid substitutions at positions corresponding to positions 237 and 279 of IgG4 (SEQ ID NO:4), wherein the amino acids at positions corresponding to positions 237 and 279 of IgG4 (SEQ ID NO:4) are replaced with lysine.

In some embodiments, the TAA-binding agent is a bispecific agent which comprises a first human IgG1 constant region with amino acid substitutions at positions corresponding to positions 253 and 292 of IgG1 (SEQ ID NO:1), wherein the amino acids are replaced with glutamate, and a second human IgG1 constant region with amino acid substitutions at positions corresponding to positions 240 and 282 of IgG1 (SEQ ID NO:1), wherein the amino acids are replaced with lysine. In some embodiments, the TAA-binding agent is a bispecific antibody which comprises a first human IgG1 constant region with amino acid substitutions at positions corresponding to positions 253 and 292 of IgG1 (SEQ ID NO:1), wherein the amino acids are replaced with aspartate, and a second human IgG1 constant region with amino acid substitutions at positions corresponding to positions 240 and 282 of IgG1 (SEQ ID NO:1), wherein the amino acids are replaced with lysine.

In some embodiments, the TAA-binding agent is a bispecific agent which comprises a first human IgG2 constant region with amino acid substitutions at positions corresponding to positions 249 and 288 of IgG2 (SEQ ID NO:2), wherein the amino acids are replaced with glutamate, and a second human IgG2 constant region with amino acid substitutions at positions corresponding to positions 236 and 278 of IgG2 (SEQ ID NO:2), wherein the amino acids are replaced with lysine. In some embodiments, the TAA-binding agent is a bispecific antibody which comprises a first human IgG2 constant region with amino acid substitutions at positions corresponding to positions 249 and 288 of IgG2 (SEQ ID NO:2), wherein the amino acids are replaced with aspartate, and a second human IgG2 constant region with amino acid substitutions at positions corresponding to positions 236 and 278 of IgG2 (SEQ ID NO:2), wherein the amino acids are replaced with lysine.

The invention provides polypeptides, including, but not limited to, antibodies that specifically bind a TAA.

Many proteins, including antibodies, contain a signal sequence that directs the transport of the proteins to various locations. Generally, signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or may be used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions as compared to a “native” or “parental” signal sequence. In some embodiments, the signal sequence of the polypeptide comprises amino acid substitutions and/or deletions that allow one cleavage site to be dominant, thereby resulting in a substantially homogeneous polypeptide with one N-terminus. In some embodiments, a signal sequence of the polypeptide affects the expression level of the polypeptide, e.g., increased expression or decreased expression.

In certain embodiments, the TAA-binding agents described herein have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, inhibit tumor growth, trigger cell death of tumor cells, enhance or increase the immunogenicity of a tumor, enhance or boost the immune response, enhance or boost the anti-tumor response, increase cytolytic activity of immune cells, increase killing of tumor cells, increase killing of tumor cells by immune cells, redirect an existing immune response to a new target, redirect an existing immune response to a tumor target, prevent metastasis of tumor cells, decrease survival of tumor cells, increase tumor cell apoptosis, or decrease survival of tumor cells.

In certain embodiments, the TAA-binding agents described herein inhibit tumor growth. In certain embodiments, a TAA-binding agent inhibits tumor growth in vivo (e.g., in a mouse model, and/or in a human having cancer). In certain embodiments, tumor growth is inhibited at least about two-fold, about three-fold, about five-fold, about ten-fold, about 50-fold, about 100-fold, or about 1000-fold as compared to a untreated tumor or a tumor treated with a control molecule.

In certain embodiments, a TAA-binding agent inhibits tumor growth in an animal model, such as a mouse model. In some embodiments, the mouse model is a mouse xenograft model. In some embodiments, the mouse model is a mouse xenograft model wherein the xenograft is a human tumor. In some embodiments, the mouse model comprises an immunocompromised mouse (e.g., NOD/SCID) wherein human tumor cells are mixed with human PBMCs and subcutaneously injected in the mouse. In such a model, the TAA-binding agent can bind the human TAA and elicit a response from the human immune cells. In some embodiments, the mouse model is a “humanized” mouse model. A general description of humanized mice includes mice engrafted with functional human cells or tissues or expressing human transgenes. In some embodiments, humanized mice develop at least a partial human immune system, including functional T-cells and B-cells. The engrafted functional human immune system is capable of T-cell and B-cell-dependent immune responses, antibody production, anti-viral responses, and allograft rejection. In some embodiments, a TAA-binding agent that binds a human TAA is used in a humanized mouse model where the mouse has been injected with a human tumor.

In certain embodiments, the TAA-binding agents reduce the tumorigenicity of a tumor in an animal model, such as a mouse model. In certain embodiments, the TAA-binding agents reduce the tumorigenicity of a tumor comprising cancer stem cells in an animal model, such as a mouse model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In some embodiments, a TAA-binding agent described herein delivers an exogenous polypeptide comprising at least one antigenic peptide to a tumor cell. In some embodiments, the TAA-binding agent specifically binds a TAA and delivers the exogenous polypeptide comprising at least one antigenic peptide to a tumor cell expressing the TAA. In some embodiments, the TAA-binding agent binds the TAA and is internalized by the tumor cell. In some embodiments, the TAA-binding agent binds the TAA, is internalized by the tumor cell, and the antibody with the exogenous polypeptide is processed by the cell. In some embodiments, the TAA-binding agent binds the TAA, is internalized by the tumor cell, and the antigenic peptide is presented on the surface of the tumor cell. In some embodiments, the TAA-binding agent binds the TAA, is internalized by the tumor cell, the antibody with the exogenous polypeptide is processed by the cell, and the antigenic peptide is presented on the surface of the tumor cell.

In some embodiments, a TAA-binding agent described herein delivers an exogenous polypeptide comprising at least one antigenic peptide to a tumor cell, wherein the antigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments, a TAA-binding agent described herein delivers an exogenous polypeptide comprising at least one antigenic peptide to a tumor cell, wherein the antigenic peptide is presented on the surface of the tumor cell, and an immune response against the tumor cell is induced. In some embodiments, the immune response against the tumor cell is enhanced. In some embodiments, the immune response against the tumor cell is increased. In some embodiments, the TAA-binding agent delivers an exogenous polypeptide comprising at least one antigenic peptide to a tumor cell, wherein the antigenic peptide is presented on the surface of the tumor cell, and tumor growth is inhibited.

In some embodiments, a TAA-binding agent described herein delivers an exogenous polypeptide comprising at least one antigenic peptide to a tumor cell, wherein the antigenic peptide is presented on the surface of the tumor cell, and T-cell killing directed against the tumor cell is induced. In some embodiments, T-cell killing directed against the tumor cell is enhanced. In some embodiments, T-cell killing directed against the tumor cell is increased.

In certain embodiments, TAA-binding agents (e.g., polypeptides and/or antibodies) described herein bind a TAA and modulate an immune response. In some embodiments, a TAA-binding agent redirects an existing immune response to a new target. In some embodiments, a TAA-binding agent redirects an existing immune response to a TAA-expressing tumor cell or cancer cell. In some embodiments, the existing immune response is an anti-viral response and the TAA-binding agent comprises an anti-viral T-cell epitope. In some embodiments, a TAA-binding agent described herein activates and/or increases an immune response. In some embodiments, a TAA-binding agent described herein activates and/or increases an immune response and redirects the immune response to a TAA-expressing cell. In some embodiments, a TAA-binding agent increases, promotes, or enhances cell-mediated immune response. In some embodiments, a TAA-binding agent increases, promotes, or enhances cell-mediated immune response and redirects the cell-mediated immune response to a TAA-expressing cell. In some embodiments, a TAA-binding agent increases, promotes, or enhances T-cell activity. In some embodiments, a TAA-binding agent increases, promotes, or enhances cytolytic T-cell (CTL) activity. In some embodiments, a TAA-binding agent increases, promotes, or enhances lymphokine-activated killer cell (LAK) activity. In some embodiments, a TAA-binding agent increases, promotes, or enhances tumor-infiltrating lymphocyte (TIF) activity. In some embodiments, a TAA-binding agent increases, promotes, or enhances tumor cell killing. In some embodiments, a TAA-binding agent increases, promotes, or enhances the inhibition of tumor growth.

In some embodiments, a TAA-binding agent induces and/or enhances a Th1 immune response. In general, a Th1 immune response includes production of interferon-gamma (IFN-γ), IL-2, and tumor necrosis factor-beta (TNF-β). In comparison, a Th2 immune response generally includes production of IL-4, IL-5, IL-6, IL-9, IL-10, and IL-13. In some embodiments, a TAA-binding agent induces and/or increases cytokine or lymphokine production. In some embodiments, the induction and/or increase in cytokine or lymphokines production is an indirect effect. In some embodiments, a TAA-binding agent reduces or inhibits regulatory T-cell (Treg) activity. In some embodiments, the reduction and/or inhibition of Treg activity is an indirect effect. In some embodiments, a TAA-binding agent reduces or inhibits regulatory myeloid-derived suppressor cell (MDSC) activity. In some embodiments, the reduction and/or inhibition of MDSC activity is an indirect effect.

In vivo and in vitro assays for determining whether a TAA-binding agent (or candidate binding agent) modulates an immune response are known in the art or are being developed. In some embodiments, a functional assay that detects T-cell activation may be used. In some embodiments, a functional assay that detects T-cell proliferation may be used. In some embodiments, a functional assay that detects NK activity may be used. In some embodiments, a functional assay that detects CTL activity may be used. In some embodiments, a functional assay that detects Treg activity may be used. In some embodiments, a functional assay that detects MDSC activity may be used. In some embodiments, a functional assay that detects production of cytokines or lymphokines or cells producing cytokines or lymphokines may be used. In some embodiments, an ELISpot assay is used to measure antigen-specific T-cell frequency. In some embodiments, an ELISpot assay is used to measure cytokine release/production and/or used to measure the number of cytokine producing cells. In some embodiments, cytokine assays are used to identify a Th1 response. In some embodiments, cytokine assays are used to identify a Th2 response. In some embodiments, cytokine assays are used to identify a Th17 response. In some embodiments, FACS analysis is used to measure activation markers on immune cells, including but not limited to, T-cells, B-cells, NK cells, macrophages, and/or myeloid cells.

In certain embodiments, a TAA-binding agent described herein has a circulating half-life in mice, rats, cynomolgus monkeys, or humans of at least about 2 hours, at least about 5 hours, at least about 10 hours, at least about 24 hours, at least about 3 days, at least about 1 week, or at least about 2 weeks. In certain embodiments, the TAA-binding agent is an IgG (e.g., IgG1, IgG2, or IgG4) antibody that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0. Known methods of increasing the circulating half-life of antibody fragments lacking the Fc region include such techniques as PEGylation.

In some embodiments described herein, the TAA-binding agents are polypeptides. In some embodiments, the polypeptides are recombinant polypeptides, natural polypeptides, or synthetic polypeptides comprising an antibody, or fragment thereof, that bind specific TAAs. It will be recognized in the art that some amino acid sequences of the invention can be varied without significant effect of the structure or function of the protein. Thus, the invention further includes variations of the polypeptides which show substantial activity or which include regions of an antibody, or fragment thereof, that binds a TAA. In some embodiments, amino acid sequence variations of TAA-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve or otherwise modulate the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{st} Edition, 2012, Pharmaceutical Press, London.

In certain embodiments, the polypeptides described herein are isolated. In certain embodiments, the polypeptides described herein are substantially pure.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies, or fragments thereof, that specifically bind a human TAA. For example, recombinant expression vectors can be replicable DNA constructs which have synthetic or cDNA-derived DNA fragments encoding a polypeptide chain of a TAA-binding agent, such as an anti-TAA antibody, or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, in situations where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The TAA-binding agents (e.g., polypeptides or antibodies) of the present invention can be expressed from one or more vectors. For example, in some embodiments, one heavy chain polypeptide is expressed by one vector, a second heavy chain polypeptide is expressed by a second vector and a light chain polypeptide is expressed by a third vector. In some embodiments, a first heavy chain polypeptide and a light chain polypeptide is expressed by one vector and a second heavy chain polypeptide is expressed by a second vector. In some embodiments, two heavy chain polypeptides are expressed by one vector and a light chain polypeptide is expressed by a second vector. In some embodiments, three polypeptides are expressed from one vector. Thus, in some embodiments, a first heavy chain polypeptide, a second heavy chain polypeptide, and a light chain polypeptide are expressed by a single vector.

Suitable host cells for expression of a TAA-binding polypeptide or antibody (or a TAA protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the TAA-binding agents described herein. In some embodiments, the cells produce the TAA-binding agents described herein. In certain embodiments, the cells produce an antibody. In some embodiments, the cells produce an antibody that binds a human TAA. In some embodiments, the cell is a hybridoma cell. In some embodiments, the cell is a mammalian cell. In some embodiments, the cell is a prokaryotic cell. In some embodiments, the cell is an eukaryotic cell.

The proteins (e.g., an antibody) produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a recombinant protein (e.g., a TAA-binding agent). Some or all of the foregoing purification steps, in various combinations, can be employed to provide a homogeneous recombinant protein.

In some embodiments, heterodimeric proteins such as bispecific antibodies are purified according the any of the methods described herein. In some embodiments, anti-TAA bispecific antibodies are isolated and/or purified using at least one chromatography step. In some embodiments, the at least one chromatography step comprises affinity chromatography. In some embodiments, the at least one chromatography step further comprises anion exchange chromatography. In some embodiments, the isolated and/or purified antibody product comprises at least 90% heterodimeric antibody. In some embodiments, the isolated and/or purified antibody product comprises at least 95%, 96%, 97%, 98% or 99% heterodimeric antibody. In some embodiments, the isolated and/or purified antibody product comprises about 100% heterodimeric antibody.

In some embodiments, a polypeptide produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the TAA-binding agent is a polypeptide that is not an antibody or does not comprise an immunoglobulin Fc region. A variety of methods for identifying and producing non-antibody polypeptides that bind with high affinity to a protein target are known in the art. In certain embodiments, phage or mammalian display technology may be used to produce and/or identify a TAA-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art. In certain embodiments, phage display technology may be used to produce and/or identify a binding polypeptide. In certain embodiments, mammalian cell display technology may be used to produce and/or identify a binding polypeptide.

Heteroconjugate molecules are also within the scope of the present invention. Heteroconjugate molecules are composed of two covalently joined polypeptides. Such molecules have, for example, been proposed to target immune cells to unwanted cells, such as tumor cells. It is also contemplated that the heteroconjugate molecules can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain embodiments, the TAA-binding agents can be used in any one of a number of conjugated (i.e. an immunoconjugate or radioconjugate) or non-conjugated forms.

In some embodiments, the TAA-binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as calicheamicins, maytansinoids, trichothenes, and CC1065, and the derivatives of these toxins that have toxin activity, can also be used. Conjugates of an antibody and cytotoxic agent may be made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Polynucleotides

In certain embodiments, the invention encompasses polynucleotides comprising polynucleotides that encode an agent described herein. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, hybridization techniques are conducted under conditions of high stringency. Conditions of high stringency are known to those of skill in the art and may include but are not limited to, (1) employ low ionic strength and high temperature for washing, for example 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

In certain embodiments, a polynucleotide comprises the coding sequence of the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide. The polynucleotides can also encode for a pro-protein which is the mature protein plus additional 5′ amino acid residues. A mature protein having a pro-sequence is a pro-protein and is an inactive form of the protein. Once the pro-sequence is cleaved an active mature protein remains.

In certain embodiments, a polynucleotide comprises the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag which can be used in conjunction with other affinity tags.

The present invention further relates to variants of the polynucleotides described herein, where the variants encode, for example, fragments, analogs, and/or derivatives.

In certain embodiments, the present invention provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a TAA-binding agent described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric molecule. In some embodiments, at least one polynucleotide variant is produced (without changing the amino acid sequence of the encoded polypeptide) to increase production of a bispecific antibody.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. Methods of Use and Pharmaceutical Compositions

The TAA-binding agents of the invention are useful in a variety of applications including, but not limited to, therapeutic treatment methods, such as the treatment of cancer. In some embodiments, the therapeutic treatment methods comprise immunotherapy. In certain embodiments, a TAA-binding agent is useful for activating, promoting, increasing, and/or enhancing an immune response, redirecting an existing immune response to a new target (e.g., TAA-expressing target), increasing the immunogenicity of a tumor, inhibiting tumor growth, reducing tumor volume, increasing tumor cell apoptosis, and/or reducing the tumorigenicity of a tumor. The methods of use may be in vitro, ex vivo, or in vivo methods.

The present invention provides methods for activating an immune response in a subject using a TAA-binding agent described herein. In some embodiments, the invention provides methods for promoting an immune response in a subject using a TAA-binding agent described herein. In some embodiments, the invention provides methods for increasing an immune response in a subject using a TAA-binding agent described herein. In some embodiments, the invention provides methods for enhancing an immune response using a TAA-binding agent. In some embodiments, the activating, promoting, increasing, and/or enhancing of an immune response comprises increasing cell-mediated immunity. In some embodiments, the activating, promoting, increasing, and/or enhancing of an immune response comprises increasing T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of Tregs. In some embodiments, the activating, promoting, increasing, and/or enhancing of an immune response comprises inhibiting or decreasing the suppressive activity of MDSCs. In some embodiments, the immune response is a result of antigenic stimulation. In some embodiments, the antigenic stimulation is a tumor cell. In some embodiments, the antigenic stimulation is cancer.

In some embodiments, the invention provides methods of activating, promoting, increasing, and/or enhancing of an immune response using a TAA-binding agent described herein. In some embodiments, a method comprises a TAA-binding agent that delivers an exogenous polypeptide comprising at least one antigenic peptide to a tumor cell. In some embodiments, a method comprises a TAA-binding agent that delivers the exogenous polypeptide comprising at least one antigenic peptide to a tumor cell expressing the TAA. In some embodiments, a method comprises a TAA-binding agent that binds the TAA and is internalized by the tumor cell. In some embodiments, a method comprises a TAA-binding agent that is internalized by a tumor cell, and the TAA-binding agent is processed by the cell. In some embodiments, a method comprises a TAA-binding agent that is internalized by a tumor cell, and an antigenic peptide is presented on the surface of the tumor cell. In some embodiments, a method comprises a TAA-binding agent that is internalized by the tumor cell, is processed by the cell, and an antigenic peptide is presented on the surface of the tumor cell.

In some embodiments, a method comprises a TAA-binding agent described herein that delivers an exogenous polypeptide comprising at least one antigenic peptide to a tumor cell, wherein the antigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments, a method comprises a TAA-binding agent described herein that delivers an exogenous polypeptide comprising at least one antigenic peptide to a tumor cell, wherein the antigenic peptide is presented on the surface of the tumor cell, and an immune response against the tumor cell is induced. In some embodiments, the immune response against the tumor cell is enhanced. In some embodiments, the immune response against the tumor cell is increased. In some embodiments, the TAA-binding agent delivers an exogenous polypeptide comprising at least one antigenic peptide to a tumor cell, wherein the antigenic peptide is presented on the surface of the tumor cell, and tumor growth is inhibited.

In some embodiments, a method comprises a TAA-binding agent described herein that delivers an exogenous polypeptide comprising at least one antigenic peptide to a tumor cell, wherein the antigenic peptide is presented on the surface of the tumor cell, and T-cell killing directed against the tumor cell is induced. In some embodiments, T-cell killing directed against the tumor cell is enhanced. In some embodiments, T-cell killing directed against the tumor cell is increased.

In some embodiments, a method of increasing an immune response in a subject comprises administering to the subject a therapeutically effective amount of a TAA-binding agent described herein, wherein the agent is an antibody that specifically binds the extracellular domain of the TAA. In some embodiments, a method of increasing an immune response in a subject comprises administering to the subject a therapeutically effective amount of a TAA-binding agent described herein, wherein the agent is an antibody that specifically binds the extracellular domain of human TAA.

The present invention provides methods of redirecting an existing immune response to a tumor. In some embodiments, a method of redirecting an existing immune response to a tumor comprises administering to a subject a therapeutically effective amount of a TAA-binding agent described herein. In some embodiments, the existing immune response in against a virus. In some embodiments, the virus is selected from the group consisting of: measles virus, varicella-zoster virus (VZV; chickenpox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the virus is varicella-zoster virus. In some embodiments, the virus is cytomegalovirus. In some embodiments, the virus is measles virus. In some embodiments, the existing immune response has been acquired after a natural viral infection. In some embodiments, the existing immune response has been acquired after vaccination against a virus. In some embodiments, the existing immune response is a cell-mediated response. In some embodiments, the existing immune response comprises cytotoxic T-cells (CTLs).

In some embodiments, a method of redirecting an existing immune response to a tumor in a subject comprises administering a fusion protein comprising (i) an antibody that specifically binds a tumor-associated antigen (TAA) and (ii) at least one antigenic peptide, wherein (a) the fusion protein is internalized by a tumor cell after binding to the TAA; (b) the antigenic peptide is processed and presented on the surface of the tumor cell associated with a MHC class I molecule; and (c) the peptide/MHC Class I complex is recognized by cytotoxic T-cells. In some embodiments, the cytotoxic T-cells are memory T-cells. In some embodiments, the memory T-cells are the result of a vaccination with the antigenic peptide. In some embodiments, the memory T-cells are the result of a natural infection with a pathogen that comprises the antigenic peptide.

The present invention provides methods of increasing the immunogenicity of a tumor. In some embodiments, a method of increasing the immunogenicity of a tumor comprises contacting the tumor or tumor cells with an effective amount of a TAA-binding agent described herein. In some embodiments, a method of increasing the immunogenicity of a tumor comprises administering to a subject a therapeutically effective amount of a TAA-binding agent described herein.

The present invention also provides methods for inhibiting growth of a tumor using a TAA-binding agent described herein. In certain embodiments, a method of inhibiting growth of a tumor comprises contacting a cell mixture with a TAA-binding agent in vitro. For example, an immortalized cell line or a cancer cell line mixed with immune cells (e.g., T-cells) is cultured in medium to which is added a test agent that binds TAA. In some embodiments, tumor cells are isolated from a patient sample such as, for example, a tissue biopsy, pleural effusion, or blood sample, mixed with immune cells (e.g., T-cells), and cultured in medium to which is added a test agent that binds TAA. In some embodiments, a TAA-binding agent increases, promotes, and/or enhances the activity of the immune cells. In some embodiments, a TAA-binding agent inhibits tumor cell growth. In some embodiments, a TAA-binding agent activates killing of the tumor cells.

In some embodiments, a method of inhibiting growth of a tumor comprises contacting the tumor or tumor cells with a TAA-binding agent described herein in vivo. In certain embodiments, contacting a tumor or tumor cell with a TAA-binding agent is undertaken in an animal model. For example, a test agent may be administered to mice which have tumors. In some embodiments, a TAA-binding agent increases, promotes, and/or enhances the activity of immune cells in the mice. In some embodiments, a TAA-binding agent inhibits tumor growth. In some embodiments, a TAA-binding agent causes a tumor to regress.

In some embodiments, a method of inhibiting growth of a tumor comprises contacting the tumor or tumor cells with a TAA-binding agent.

In certain embodiments, a method of inhibiting growth of a tumor comprises administering to a subject a therapeutically effective amount of a TAA-binding agent described herein. In certain embodiments, the subject is a human. In certain embodiments, the subject has a tumor or the subject had a tumor which was at least partially removed.

In some embodiments, a method of inhibiting growth of a tumor comprises redirecting an existing immune response to a new target, comprising administering to a subject a therapeutically effective amount of a TAA-binding agent, wherein the existing immune response is against an antigenic peptide delivered to the tumor cell by the TAA-binding agent. In some embodiments, the antigenic peptide is a T-cell epitope. In some embodiments, the antigenic peptide is a CTL epitope. In some embodiments, the redirected immune response is a CTL response.

In addition, the invention provides a method of inhibiting growth of a tumor in a subject, comprising administering to the subject a therapeutically effective amount of a TAA-binding agent described herein. In certain embodiments, the tumor comprises cancer stem cells. In certain embodiments, the frequency of cancer stem cells in the tumor is reduced by administration of the TAA-binding agent. In some embodiments, a method of reducing the frequency of cancer stem cells in a tumor in a subject, comprising administering to the subject a therapeutically effective amount of a TAA-binding agent is provided.

In addition, the invention provides a method of reducing the tumorigenicity of a tumor in a subject, comprising administering to the subject a therapeutically effective amount of a TAA-binding agent described herein. In certain embodiments, the tumor comprises cancer stem cells. In some embodiments, the tumorigenicity of a tumor is reduced by reducing the frequency of cancer stem cells in the tumor. In some embodiments, the methods comprise using the TAA-binding agents described herein. In certain embodiments, the frequency of cancer stem cells in the tumor is reduced by administration of a TAA-binding agent described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

In some embodiments, a method of inhibiting tumor growth in a subject comprises administering to the subject a therapeutically effective amount of a TAA-binding agent described herein, wherein the TAA is B7-H4 and the tumor is a breast tumor. In some embodiments, a method of inhibiting tumor growth in a subject comprises administering to the subject a therapeutically effective amount of a TAA-binding agent described herein, wherein the TAA is B7-H4 and the tumor is an ovarian tumor.

The present invention further provides methods for treating cancer in a subject comprising administering to the subject a therapeutically effective amount of a TAA-binding agent described herein. In some embodiments, a TAA-binding agent binds the human TAA and inhibits or reduces growth of the cancer. In some embodiments, a TAA-binding agent binds the human TAA on the cell surface of a tumor cell, delivers a T-cell epitope to the tumor cell, and activates an immune response targeting the tumor cell.

In some embodiments, a method of treating cancer comprises redirecting an existing immune response to a new target, the method comprising administering to a subject a therapeutically effective amount of a TAA-binding agent, wherein the existing immune response is against an antigenic peptide delivered to the cancer cell by the TAA-binding agent. In some embodiments, the antigenic peptide is a T-cell epitope. In some embodiments, the antigenic peptide is a CTL epitope. In some embodiments, the redirected immune response is a CTL response.

The present invention provides for methods of treating cancer comprising administering to a subject a therapeutically effective amount of a TAA-binding agent described herein (e.g., a subject in need of treatment). In certain embodiments, the subject is a human. In certain embodiments, the subject has a cancerous tumor. In certain embodiments, the subject has had a tumor at least partially removed.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer. In some embodiments, the cancer comprises a solid tumor.

In some embodiments, a method of treating cancer in a subject comprises administering to the subject a therapeutically effective amount of a TAA-binding agent described herein, wherein the TAA is B7-H4 and the cancer is breast cancer. In some embodiments, a method of treating cancer in a subject comprises administering to the subject a therapeutically effective amount of a TAA-binding agent described herein, wherein the TAA is B7-H4 and the cancer is ovarian cancer.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a TAA-binding agent described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that may be administered in combination with the TAA-binding agents described herein include chemotherapeutic agents. Thus, in some embodiments, the method or treatment involves the administration of an agent of the present invention in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present invention with a small molecule that acts as an inhibitor against tumor-associated antigens including, but not limited to, EGFR, HER2 (ErbB2), and/or VEGF. In some embodiments, an agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is an agent that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the RSPO/LGR pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of an agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the TAA-binding agent can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, PlGF, gamma-IFN, IL-1, IL-2, IL-3, IL-4, IL-5, IL-6, IL-7, IL-12, IL-15, and IL-18.

In some embodiments, treatment with a TAA-binding agent described herein can be accompanied by surgical removal of tumors, removal of cancer cells, or any other surgical therapy deemed necessary by a treating physician.

In certain embodiments, treatment involves the administration of a TAA-binding agent of the present invention in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a TAA-binding agent described herein and at least one additional therapeutic agent may be administered in any order or concurrently. In some embodiments, the agent will be administered to patients that have previously undergone treatment with a second therapeutic agent. In certain other embodiments, the TAA-binding agent and a second therapeutic agent will be administered substantially simultaneously or concurrently. For example, a subject may be given an agent while undergoing a course of treatment with a second therapeutic agent (e.g., chemotherapy). In certain embodiments, a TAA-binding agent will be administered within 1 year of the treatment with a second therapeutic agent. In certain alternative embodiments, a TAA-binding agent will be administered within 10, 8, 6, 4, or 2 months of any treatment with a second therapeutic agent. In certain other embodiments, a TAA-binding agent will be administered within 4, 3, 2, or 1 weeks of any treatment with a second therapeutic agent. In some embodiments, an agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of a TAA-binding agent of the present invention depends on the type of disease to be treated, the severity and course of the disease, the responsiveness of the disease, whether the agent is administered for therapeutic or preventative purposes, previous therapy, the patient's clinical history, and so on, all at the discretion of the treating physician. The TAA-binding agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the agent is from about 0.1 mg to about 20 mg/kg of body weight. In some embodiments, the dosage of the agent is about 0.5 mg/kg of body weight. In some embodiments, the dosage of the agent is about 1 mg/kg of body weight. In some embodiments, the dosage of the agent is about 1.5 mg/kg of body weight. In some embodiments, the dosage of the agent is about 2 mg/kg of body weight. In some embodiments, the dosage of the agent is about 2.5 mg/kg of body weight. In some embodiments, the dosage of the agent is about 5 mg/kg of body weight. In some embodiments, the dosage of the agent is about 7.5 mg/kg of body weight. In some embodiments, the dosage of the agent is about 10 mg/kg of body weight. In some embodiments, the dosage of the agent is about 12.5 mg/kg of body weight. In some embodiments, the dosage of the agent is about 15 mg/kg of body weight.

In some embodiments, a TAA-binding agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present invention provides methods of administering to a subject the TAA-binding agents described herein comprising using an intermittent dosing strategy for administering one or more agents, which may reduce side effects and/or toxicities associated with administration of an agent, chemotherapeutic agent, etc. In some embodiments, a method for treating cancer in a human subject comprises administering to the subject a therapeutically effective dose of an agent in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of an agent in combination with a therapeutically effective dose of a second immunotherapeutic agent, wherein one or both of the agents are administered according to an intermittent dosing strategy. In some embodiments, the intermittent dosing strategy comprises administering an initial dose of an agent to the subject, and administering subsequent doses of the agent about once every 2 weeks. In some embodiments, the intermittent dosing strategy comprises administering an initial dose of an agent to the subject, and administering subsequent doses of the agent about once every 3 weeks. In some embodiments, the intermittent dosing strategy comprises administering an initial dose of an agent to the subject, and administering subsequent doses of the agent about once every 4 weeks. In some embodiments, the agent is administered using an intermittent dosing strategy and the additional therapeutic agent is administered weekly.

The present invention provides compositions comprising the TAA-binding agents described herein. The present invention also provides pharmaceutical compositions comprising the TAA-binding agents described herein and a pharmaceutically acceptable vehicle. In some embodiments, the pharmaceutical compositions find use in immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified antibody or agent of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{st} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The TAA-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include an agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the TAA-binding agents described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### V. Screening

The present invention provides screening methods to identify TAA-binding agents that modulate the immune response. In some embodiments, the present invention provides methods for screening candidate agents, including but not limited to, polypeptides, antibodies, peptides, peptidomimetics, small molecules, compounds, or other drugs, which modulate the immune response.

In some embodiments, a method of screening for a candidate TAA-binding agent that modulates the immune response comprises determining if the agent has an effect on immune cells. In some embodiments, a method of screening for a candidate TAA-binding agent that modulates the immune response comprises determining if the agent is capable of increasing the activity of immune cells. In some embodiments, a method of screening for a candidate TAA-binding agent that modulates the immune response comprises determining if the agent is capable of increasing the activity of cytolytic cells, such as CTLs. In some embodiments, a method of screening for a candidate TAA-binding agent that modulates the immune response comprises determining if the agent is capable of decreasing the activity of immune suppressor cells, such as Tregs or MDSCs.

### VI. Kits Comprising Agents Described Herein

The present invention provides kits that comprise the TAA-binding agents described herein and that can be used to perform the methods described herein. In certain embodiments, a kit comprises at least one purified TAA-binding agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed TAA-binding agents of the present invention can be readily incorporated into one of the established kit formats which are well known in the art.

Further provided are kits that comprise a TAA-binding agent as well as at least one additional therapeutic agent. In certain embodiments, the second (or more) therapeutic agent is a chemotherapeutic agent. In certain embodiments, the second (or more) therapeutic agent is an antibody.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Generation of Monoclonal Antibodies

Antibodies are generated against tumor-associated antigens (TAAs) using standard techniques. For example, mice are immunized with the extracellular domain or a fragment thereof of a human TAA. Sera from individual mice are screened against the TAA using flow cytometry approximately 70 days after initial immunization. The mouse with the highest antibody titer is selected for a final antigen boost after which spleen cells are isolated for hybridoma production. SP2/0 cells are used as fusion partners for the mouse spleen cells. Cells are plated at 1 cell per well in 96 well plates, and the supernatants are screened against the target TAA using flow cytometry (FACS).

For FACS screening of anti-TAA antibodies, a chimeric fusion protein comprising the extracellular domain of a TAA is constructed which allows cell surface expression of the TAA (FLAG-TAA-CD4TM-GFP) and transfected into HEK-293 cells. After 48 hours, transfected cells are suspended in ice cold PBS containing 2% FBS and incubated on ice in the presence of 50 μl of hybridoma supernatants for 30 minutes. A second incubation with 100 μl PE-conjugated anti-Fc secondary antibody is performed to detect cells bound by antibody. Cells are incubated with an anti-FLAG antibody (Sigma-Aldrich) as a positive control and an anti-PE antibody as a negative control. The cells are analyzed on a flow cytometry instrument and the data are processed using specialized software.

Hybridomas are identified that produce antibodies that bind the TAA and several are selected. The nucleotide sequences of the heavy chain variable region and the light chain variable region of these antibodies are determined. The predicted amino acid sequences and CDRs are determined.

The antibodies are humanized using standard techniques known to those of skill in the art.

Antibodies are characterized by flow cytometry analysis for binding affinity. As described above, a TAA-GFP construct is transfected into HEK-293 cells. After 48 hours, transfected cells are suspended in ice cold HBSS containing 2% FBS and incubated on ice in the presence of antibodies at a range of concentrations. A second incubation with 100 μl APC-conjugated anti-mouse or anti-human Fc secondary antibody is performed to detect cells bound by antibody. The cells are analyzed on a FACS instrument and the data is processed using specialized software.

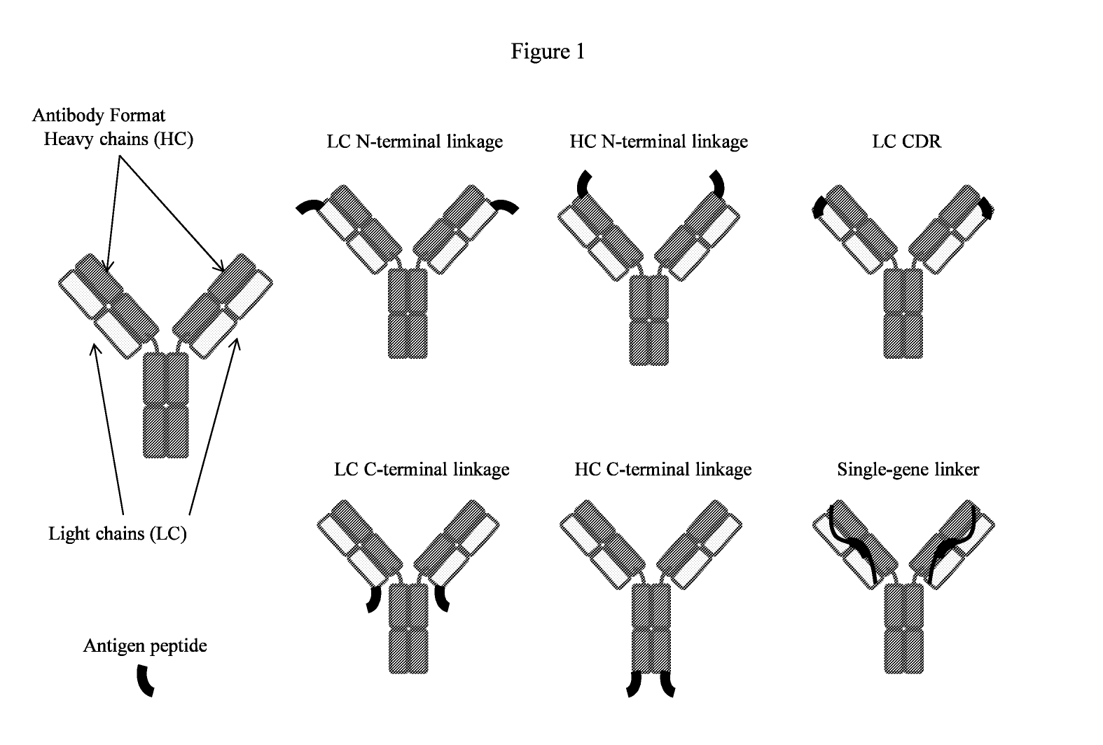

The antibodies can be modified or re-engineered to comprise at least one exogenous polypeptide. The exogenous polypeptide can comprise one or more antigenic peptides. In some embodiments, the antigenic peptide comprises a T-cell epitope. In some embodiments, the T-cell epitope is a MHC class I-restricted T-cell epitope. In some embodiments, the T-cell epitope is a CD8+ T-cell epitope. The exogenous polypeptide can comprise one or more antigenic viral peptides. The exogenous polypeptide (e.g., antigenic peptide) can be inserted or linked to the antibody in a variety of ways. The exogenous polypeptide can be linked at the amino-terminal end of the immunoglobulin light chain variable region. The exogenous polypeptide can be linked at the amino-terminal end of the immunoglobulin heavy chain variable region. The exogenous polypeptide can be linked at the carboxyl-terminal end of the immunoglobulin light chain. The exogenous polypeptide can be linked at the carboxyl-terminal end of the immunoglobulin heavy chain. The exogenous polypeptide can be inserted within the light chain variable region. The exogenous polypeptide can be inserted within a CDR of the light chain variable region. The exogenous polypeptide can be inserted within a CDR of the heavy chain variable region. The exogenous polypeptide can be inserted within a framework region of the light chain variable region. The exogenous polypeptide can be inserted within a framework region of the heavy chain variable region. The antibody can be a single chain antibody comprising an amino acid sequence linking the light chain to the heavy chain. The exogenous polypeptide can be inserted within the linker sequence of a single chain antibody. Representative diagrams are shown in FIG. 1. It should be understood that the diagrams of FIG. 1 are a non-limiting series of the possible formats for placement of an exogenous polypeptide within an antibody.

### Example 2

### Antigen Presentation by Tumor Cells

The ability of tumor cells to present an antigenic peptide to T-cells was investigated. A proof-of-concept study was set up with four different tumor cell lines to examine if the cells could present an antigen to T-cells and elicit a response. In these studies the T-cell hybridoma B3Z86/90.14 (B3Z) was used as the responding T-cell. B3Z cells stably express a T-cell receptor (TCR) that is specific for a peptide derived from chicken ovalbumin (OVA₂₅₇₋₂₆₄; SIINFEKL (SEQ ID NO:5)) and which is presented in the context of MHC class I H-2K^{b}. B3Z cells secrete IL-2 in response to the TCR binding the OVA peptide SIINFEKL-MHC complex. B3Z cells were maintained in RPMI 1640 with GlutaMAX supplemented with 10% heat-inactivated FBS, 1 mM sodium pyruvate, 50 μM 2-mercaptoethanol, 100U/ml penicillin and 100 μg/ml streptomycin. The tumor cell lines studied were B16F10, LL/2 (LLC1), MC38, and KP_LUN31. B16F10 is a mouse skin melanoma cell line which is maintained in DMEM with GlutaMAX supplemented with 10% heat-inactivated FBS, 100U/ml penicillin and 100 μg/ml streptomycin. LL/2 (LLC1) is a mouse Lewis lung carcinoma cell line which is maintained in DMEM with GlutaMAX supplemented with 10% heat-inactivated FBS, 100U/ml penicillin and 100 μg/ml streptomycin. MC38 is a mouse colon carcinoma cell line which is maintained in DMEM with GlutaMAX supplemented with 10% heat-inactivated FBS, 50 μM 2-mercaptoethanol, 1% non-essential amino acids, 100U/ml penicillin and 100 μg/ml streptomycin. KP_LUN31 is a mouse non-small cell lung cancer (NSCLC) model established at OncoMed. This tumor line was observed to have a poorly differentiated morphology, to have a very short tumor latency in vivo, and to be highly metastatic. These tumor cells are maintained in DMEM-F12 supplemented with 5% heat-inactivated FBS, 35 μg/ml bovine pituitary extract, 1×N2 supplement, 20 ng/ml mEGF, 20 ng/ml mFGF, 100U/ml penicillin, and 100 μg/ml streptomycin.

Tumor cells (1×10⁵/well) were dispensed in a 96-well, flat-bottom plate. After 7 hours, soluble OVA was added to wells in 2-fold dilution concentrations (10 to 0.0625 mg/ml). The plate was incubated for 16 hrs at 37° C. to allow for antigen uptake, processing, and presentation. Cells were washed and mildly fixed with 0.2% paraformaldehyde for 10 minutes at room temperature. Cells were washed again and B3Z T-cells (1×10⁵/well) were added to each well. The co-cultured cells were incubated for 48 hrs at 37° C. and supernatants were harvested. Each sample was done in triplicate and controls included medium only, B3Z cell only, or tumor cell only samples.

The supernatants were analyzed for IL-2 production using a commercially available ELISA kit (mouse IL-2 ELISA Ready-SET-Go!, eBioscience) following the manufacturer's instructions. Briefly, a high affinity-binding 96-well plate was coated with a capture antibody specific for mouse IL-2 and incubated overnight. The plate was blocked with ELISA diluent for 1 hr at room temperature. 100 ul/well of supernatants and controls were added to the plate and incubated for 2 hrs at room temperature or overnight at 4° C. 100 ul/well of detection antibody was added to each well and incubated for 1 hr. Avidin-HRP was added to each well, the plate was incubated for 30 minutes, and TMB solution was added to each well for color development. After 15 minutes, the reaction was stopped and the plate was read at 450 nm.

As shown in Table 1, all four tumor cell lines were able to induce IL-2 production from the B3Z T-cells. KP_LUN31 and B16F10 cells induced a strong response in a concentration-dependent manner and at low concentrations of OVA. In contrast, MC38 cells required higher concentrations of OVA to induce an IL-2 response from the B3Z cells, although the response was significant and concentration-dependent from 2.5 to 10 mg/ml. LL2 cells induced only a weak response and only at the higher concentrations of OVA.

TABLE 1

| | OVA (mg/ml) | | | | | | |
|---|---|---|---|---|---|---|---|
| | 0.125 | 0.25 | 0.5 | 1.0 | 2.5 | 5.0 | 10 |
| | IL-2 (pg/ml) | | | | | | |
| KP_LUN31 | 22 | 68 | 247 | 483 | 913 | | |
| B16F10 | NS | 22 | 76 | 323 | 589 | | |
| MC38 | NS | NS | NS | NS | 54 | 138 | 338 |
| LL2 | NS | NS | NS | NS | 11 | 26 | 33 |

NS = not significant with respect to control cells

These results suggested that tumor cells can present antigen to T-cells and have the antigen recognized by the appropriate TCR.

To establish that the B3Z T-cell activation was due to presentation by the tumor cells, parallel experiments were conducted in the presence of an irreversible proteasome inhibitor. Proteasome inhibitors inhibit intracellular processing of proteins and generation of peptides and therefore reduce formation of peptide-MHC class I complexes. Without the peptide-MHC class I complexes on the surface of presenting cells (i.e., tumor cells), the TCRs are not engaged and T-cells are not activated.

KP_LUN31 tumor cells (1×10⁵/well) were dispensed into a 96-well, flat-bottom plate. After 7 hours, soluble OVA was added at a concentration of 500 μg/ml or 250 μg/ml in the presence of the proteasome inhibitor lactacystin at a concentration of 5, 10 or 20 μM. As described above, the plate was incubated for 16 hrs at 37° C. and the cells were mildly fixed with 0.2% paraformaldehyde for 10 minutes at room temperature. B3Z T-cells (1×10⁵/well) were added to each well, the co-cultured cells were incubated for 48 hrs at 37° C., and supernatants were harvested. IL-2 was assayed as described above with a mouse IL-2 ELISA kit.

To establish that the uptake of soluble OVA by the tumor cells was receptor-mediated, parallel experiments were conducted in the presence of a scavenger receptor inhibitor. Scavenger receptor inhibitors generally block uptake for soluble proteins such as OVA and abrogate receptor-mediated endocytosis, processing, and presentation on the cell surface.

KP_LUN31 tumor cells (1×10⁵/well) were dispensed into a 96-well, flat-bottom plate. After 7 hours, soluble OVA was added at a concentration of 500 μg/ml or 250 μg/ml in the presence of the scavenger receptor inhibitor polyinosinic acid (Poly(I)) at a concentration of 200, 400 or 800 μg/ml. As described above, the plate was incubated for 16 hrs at 37° C. and the cells were mildly fixed with 0.2% paraformaldehyde for 10 minutes at room temperature. B3Z T-cells (1×10⁵/well) were added to each well, the co-cultured cells were incubated for 48 hrs at 37° C., and supernatants were harvested. IL-2 was assayed as described above with a mouse IL-2 ELISA kit.

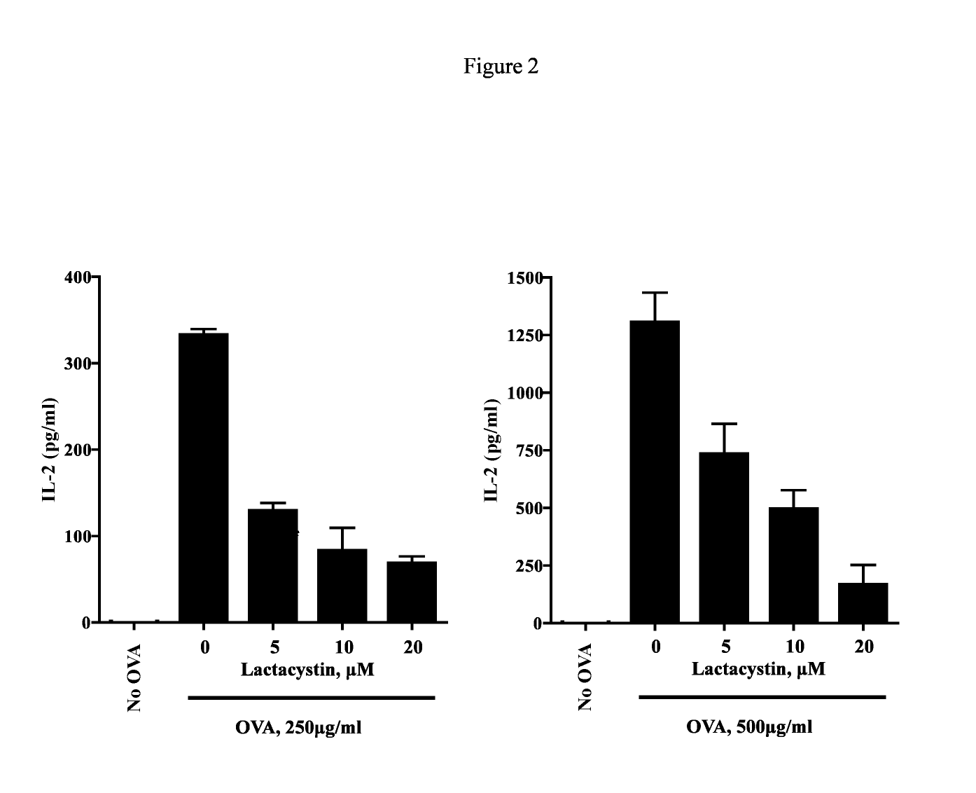

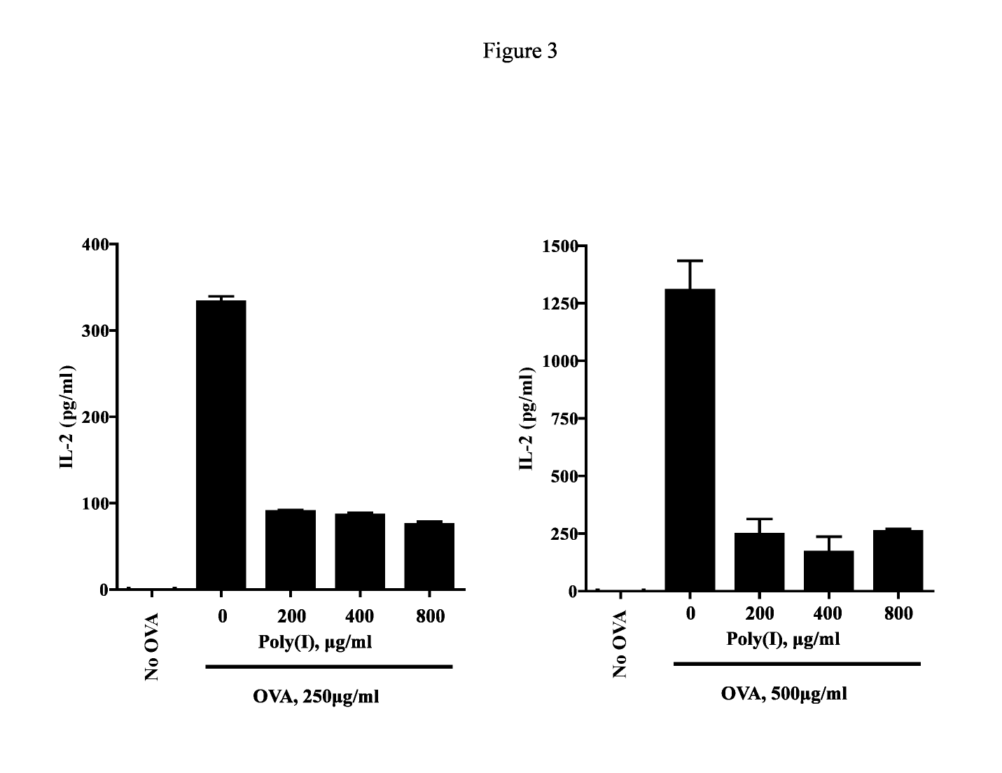

As shown in FIG. 2, the presence of lactacystin reduced IL-2 production in a concentration-dependent manner and at both OVA concentrations. In addition, the presence of Poly(I) strongly inhibited IL-2 production at both OVA concentrations (FIG. 3). These results suggest that the B3Z T-cells were activated by OVA peptides generated intracellularly and that the uptake of the soluble OVA was receptor-mediated.

### Example 3

Antigen Presentation after Targeted Antibody Binding

A proof-of-concept study was set up to determine if antigen presentation by a tumor cell can be achieved after targeted antibody binding where an antigenic peptide is delivered to the tumor cell as part of an antibody. The in vitro model system consisted of (a) cancer cells expressing mouse RSPO2 on the cell surface, (b) an anti-RSPO2 antibody comprising the OVA peptide SIINFEKL, and (c) the B3Z T-cell line that specifically becomes activated when the OVA peptide SIINFEKL is presented in the context of a MHC class I molecule.

B16F10 and KP_LUN31 tumor cells were determined to not express any detectable endogenous RSPO2 (data not shown). Stable cell lines expressing mouse RSPO2 on the cell surface were generated. B16F10 or KP_LUN31 cells (2×10⁵ cells/well) were seeded in a 6-well plate and infected with a lentivirus construct expressing a fragment of mouse RSPO2. The construct comprises the mouse RSPO2 furin-like domain 1 and furin-like domain 2 linked to a mouse CD4 transmembrane domain for cell surface expression. Cells were infected at a MOI of 55 for the B16F10 cells and at a MOI of 2.5 for KP_LUN31 cells. 8 μg/ml POLYBRENE was added during infection to increase transduction efficiency. 72 hrs after infection, cells were trypsinized, stained with an anti-RSPO2 antibody, and RSPO2 expression on the cell surface was detected by flow cytometry. RSPO2-positive cells were sorted using an ARIA 2 FACS instrument (BD Biosciences) and propagated in culture to establish stable cell lines.

The anti-RSPO2 antibody comprising the OVA peptide was generated from the anti-RSPO2 antibody 130M23 (described in U.S. Pat. No. 8,802,097). The 130M23 antibody was re-engineered as a single-chain antibody with a linker between the light chain and the variable domain of the heavy chain. The OVA peptide SIINFEKL (SEQ ID NO:5) was embedded within the linker sequence and did not interfere with binding of the antibody to RSPO2. This antibody is referred to herein as 130M23-SIINFEKL, or alternatively, 130M29.

RSPO2-expressing B16F10 and RSPO2-expressing KP_LUN31 tumor cells (1×10⁵ cells/well) as well as parental cell lines were plated in 96-well, flat-bottom plates. Seven hours after cell seeding, cells were incubated with anti-RSPO2 antibody 130M23 (200n/ml), 130M23-SIINFEKL (20n/ml or 200m/ml), or soluble OVA (100n/ml or 200m/ml). Cells were incubated for 16 hrs at 37° C. to allow for antibody binding, internalization, processing, and presentation. Cells were washed and mildly fixed with 0.2% paraformaldehyde for 10 min at room temperature. B3Z T-cells (1×10⁵/well) were added to each well, the co-cultured cells were incubated for 48 hrs at 37° C., and supernatants were harvested and analyzed for production of IL-2 as described above. Each sample was done in triplicate and controls included medium only, B3Z cells only, or tumor cells only samples.

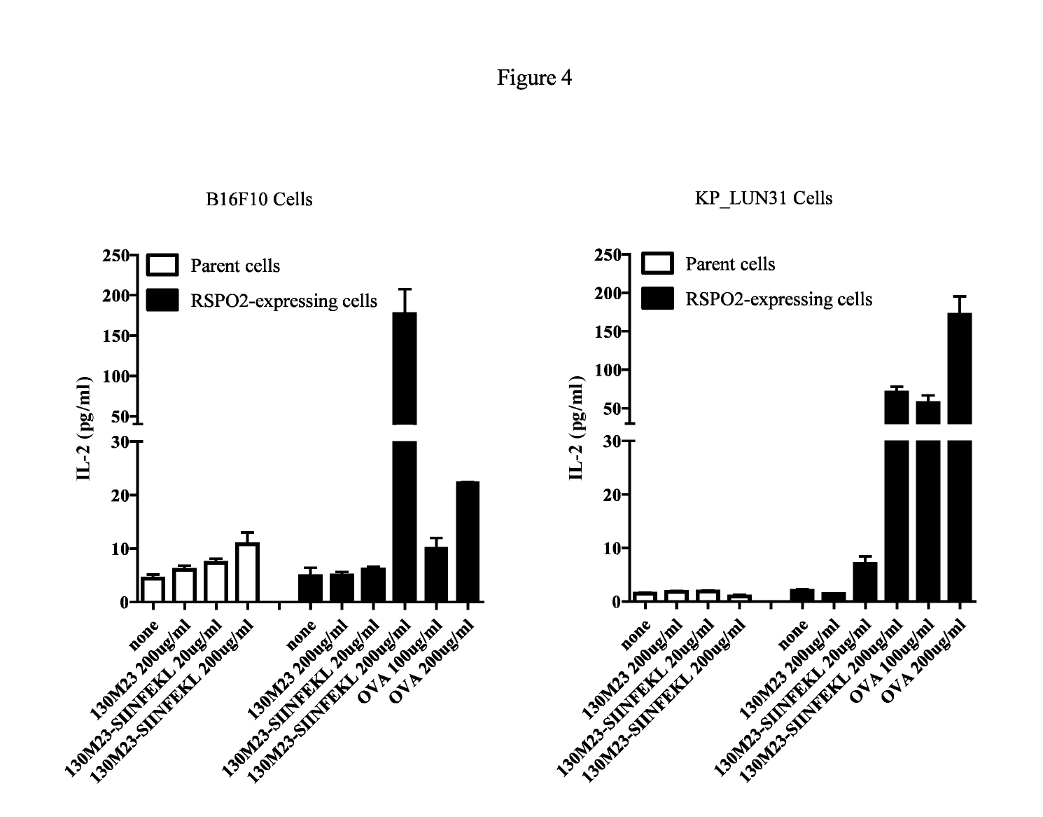

As shown in FIG. 4, RSPO2-expressing B16F10 and KP_LUN31 cell lines were able to activate B3Z T-cells after binding of the 130M23-SIINFEKL antibody to its cell surface target. The production of IL-2 by the B3Z T-cells indicated that the RSPO2-expressing tumors had internalized the 130M23-SIINFEKL antibody, processed the OVA peptide SIINFEKL, and presented it on the cell surface in a proper MHC class I complex. The results were dependent on specific antibody recognition and binding to RSPO2 since the 130M23-SIINFEKL antibody elicited no IL-2 production from the parent cells which did not express RSPO2. Importantly, the 130M23 antibody (without an OVA peptide) did not activate the B3Z T-cells. No IL-2 was produced by B3Z T-cells when the parent 130M23 antibody bound RSPO2 on the cell surface of the tumor cells. In the B16F10 cells, the amount of IL-2 secretion was higher with activation by the 130M23-SIINFEKL antibody than that observed with soluble OVA. In the KP_LUN31 cells the amount of IL-2 production was similar with the 130M23-SIINFEKL antibody and soluble OVA.

Overall these data demonstrate that tumor cells can successfully present antigenic peptides to T-cells via delivery of the antigen to the tumor cell by specific antibody-target binding.

### Example 4

T-Cell Cytotoxicity after Targeted Antibody Binding

For T-cell cytotoxicity assays, cells were harvested from the spleens of OT-I mice (Jackson Laboratories). The OT-I transgenic mouse line produces MHC class I-restricted, ovalbumin-specific CD8+ T-cells (Clarke et al., 2000, Immunology and Cell Biology, 78:110-117). Splenocytes were cultured in RPMI media with 5% heat-inactivated FBS, 100U/ml penicillin, and 100 μg/ml streptomycin and supplemented with 1 μg/ml OVA peptide SIINFEKL (SEQ ID NO:5). The cells were cultured for 5 days at 37° C., harvested, counted, and used as the effector cells in cytotoxicity assays with B16F10 or RSPO2-expressing B16F10 tumor cells as targets.

RSPO2-expressing B16F10 and B16F10 tumor cells (1×10⁵ cells/well) were plated in a 96-well, flat-bottom plate. Seven hours after cell seeding, cells were incubated with anti-RSPO2 antibody 130M23 (200 μg/ml), 130M23-SIINFEKL (200 μg/ml), or media only. Cells were incubated for 16 hrs at 37° C. to allow for antibody binding, internalization, processing, and presentation. The target tumor cells were trypsinized, counted, and labeled with 1504 calcein AM (Life Technologies) for 30 minutes at 37° C. Cells were washed and plated in a V-bottom 96-well plate (10,000 cells/well). Stimulated T-cells isolated from OT-I mice (described above) were combined with the target cells at an effector:target (E:T) ratio of 50:1, 25:1, or 12.5:1. Following a 3.5 hour incubation at 37° C., cell-free supernatants were harvested and calcein release was quantified on a fluorometer at an excitation of 485 nm and an emission of 535 nm. The percentage of specific cell lysis was determined as: % specific lysis=100×(ER−SR)/(MR−SR), where ER, SR, and MR represent experimental, spontaneous, and maximum calcein release, respectively. Spontaneous release is the fluorescence emitted by target cells incubated in media alone (i.e., in the absence of effector cells), while maximum release is determined by lysing target cells with an equal volume of 2% Triton X.

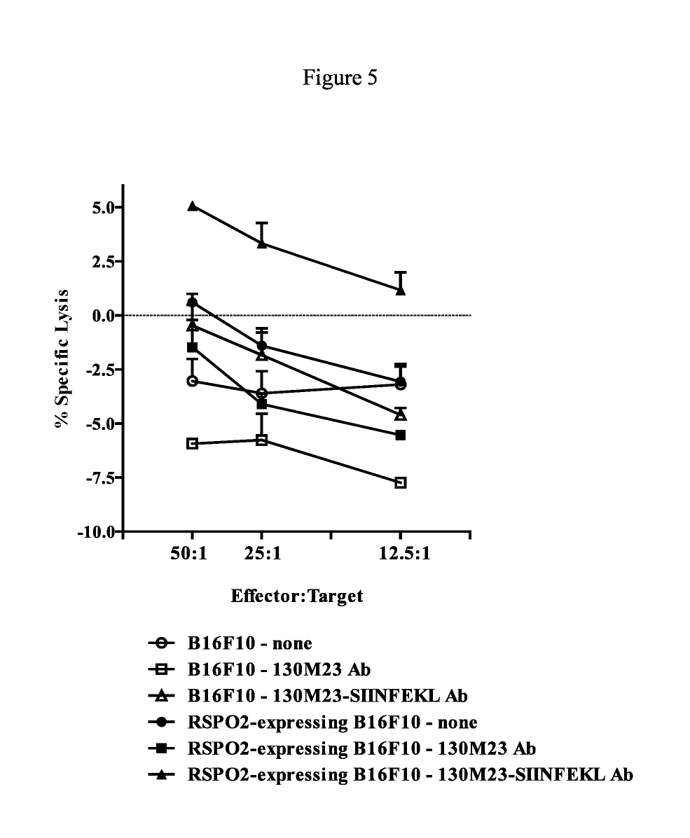

As shown in FIG. 5, RSPO2-expressing B16F10 targets that had been incubated with the 130M23-SIINFEKL antibody were killed by the OVA-specific T-cells at every E:T ratio tested and in an effector dose-dependent manner. The killing was specific for targets presenting the OVA peptide because there was no detectable killing of the RSPO2-expressing B16F10 cells that had been incubated with only the anti-RSPO2 antibody. Also, there was no detectable killing of B16F10 cells (not expressing RSPO2) that had been incubated with the anti-RSPO2-SIINFEKL antibody.

Overall these data demonstrate that tumor cells presenting antigenic peptides after delivery of the antigen to the tumor cell by specific antibody-target binding are recognized by OVA-specific cytotoxic T-cells. This is further evidence that an antigenic peptide can be delivered to a tumor cell, processed and presented in an appropriate manner and that the peptide/MHC complex is specifically recognized by a peptide specific cytotoxic T-cell.

### Example 5

Antigen Presentation after Targeted Antibody Binding

Two additional in vitro models were established with MC38 and TC1 tumor cells. MC38 is a tumor cell line derived from a primary mouse colon carcinoma and TC1 is a tumor cell line derived from a mouse lung carcinoma. As described in Example 3, stable cell lines expressing mouse RSPO2 on the cell surface were generated. MC38 or TC1 cells (2×10⁵ cells/well) were seeded in a 6-well plate and infected with a lentivirus construct expressing a fragment of mouse RSPO2 comprising the furin-like domain 1 and furin-like domain 2 linked to a mouse CD4 transmembrane domain for cell surface expression. Cells were infected at a MOI of 10 and 8 μg/ml POLYBRENE was added during infection to increase transduction efficiency. 72 hrs after infection, cells were trypsinized, stained with an anti-RSPO2 antibody, and RSPO2 expression on the cell surface was detected by flow cytometry. RSPO2-positive cells were sorted using an ARIA2 FACS instrument (BD Pharmingen). Single cell clones were propagated in culture to establish stable cell lines.

RSPO2-expressing MC38 tumor cells (2×10⁴ or 5×10⁴ cells/well) as well as parental cells were plated in collagen-coated 96-well, flat-bottom plates. Seven hours after cell seeding, cells were incubated with anti-RSPO2 antibody 130M23 (50 μg/ml), 130M23-SIINFEKL (25, 2.5, 0.25, or 0.025 μg/ml), or no antibody. RSPO2-expressing TC1 tumor cells (1×10⁴ cells/well) as well as parental cells were plated in 96-well, flat-bottom plates. Seven hours after cell seeding, cells were incubated with anti-RSPO2 antibody 130M23 (2 μg/ml), 130M23-SIINFEKL (2 or 0.2 μg/ml), or no antibody. Cells were incubated for 16 hrs at 37° C. to allow for antibody binding, internalization, processing, and presentation. Cells were washed and mildly fixed with 0.2% paraformaldehyde for 10 min at room temperature. B3Z T-cells (1×10⁵/well) were added to each well, the co-cultured cells were incubated for 48 hrs at 37° C., and supernatants were harvested and analyzed for production of IL-2 as described above.

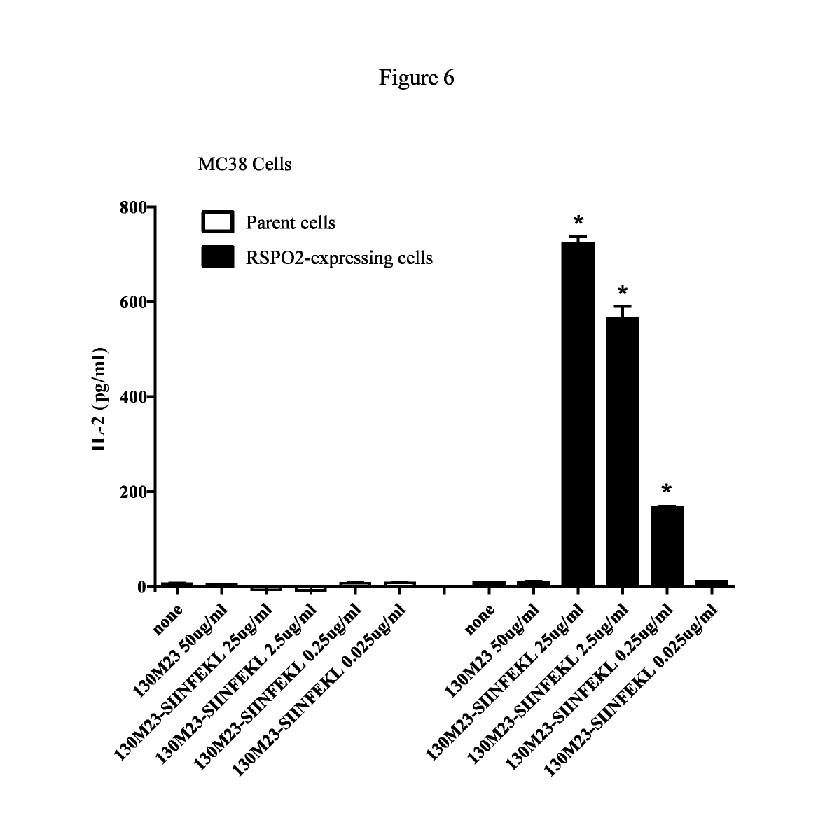

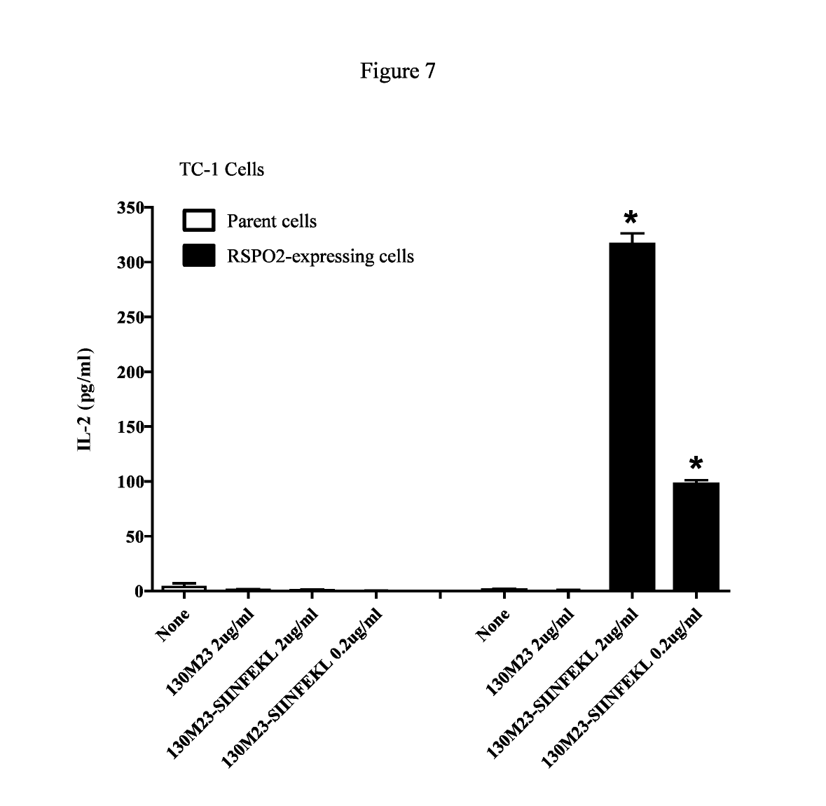

As shown in FIGS. 6 and 7, RSPO2-expressing MC38 and RSPO2-expressing TC1 cell lines were able to activate B3Z T-cells after binding of the 130M23-SIINFEKL antibody to its cell surface target. As observed with the B16F10 and KP_LUN31 cell lines, the production of IL-2 by the B3Z T-cells indicated that the RSPO2-expressing tumors had internalized the 130M23-SIINFEKL antibody, processed the OVA peptide SIINFEKL, and presented it on the cell surface in a proper MHC class I complex. Production of IL-2 by the B3Z T-cells was concentration dependent in both of the RSPO2-expressing MC38 cell and TC1 cell models. Importantly, IL-2 was produced with low amounts of antigen/antibody (e.g., 2.5 μg/ml or 2 μg/ml). Overall these data further support that different tumor type cells can successfully present antigenic peptides to T-cells via delivery of the antigen to the tumor cell by specific antibody-target binding.

### Example 6

### Effect of Levels of Cell Surface Expression of Target (TAA)

Studies were done to determine if the level of target on the tumor cell surface correlated with IL-2 production after antigen-presentation. Individual cell clones from the generation of the RSPO2-expressing MC38 cell lines were characterized for cell surface expression of RSPO2. The cell surface expression of RSPO2 was characterized by flow cytometry on five individual clones (clones #6, 9, 13, 14, and 19) and a transfection pool. These cells were tested in a presentation assay as described in Examples 3 and 5.

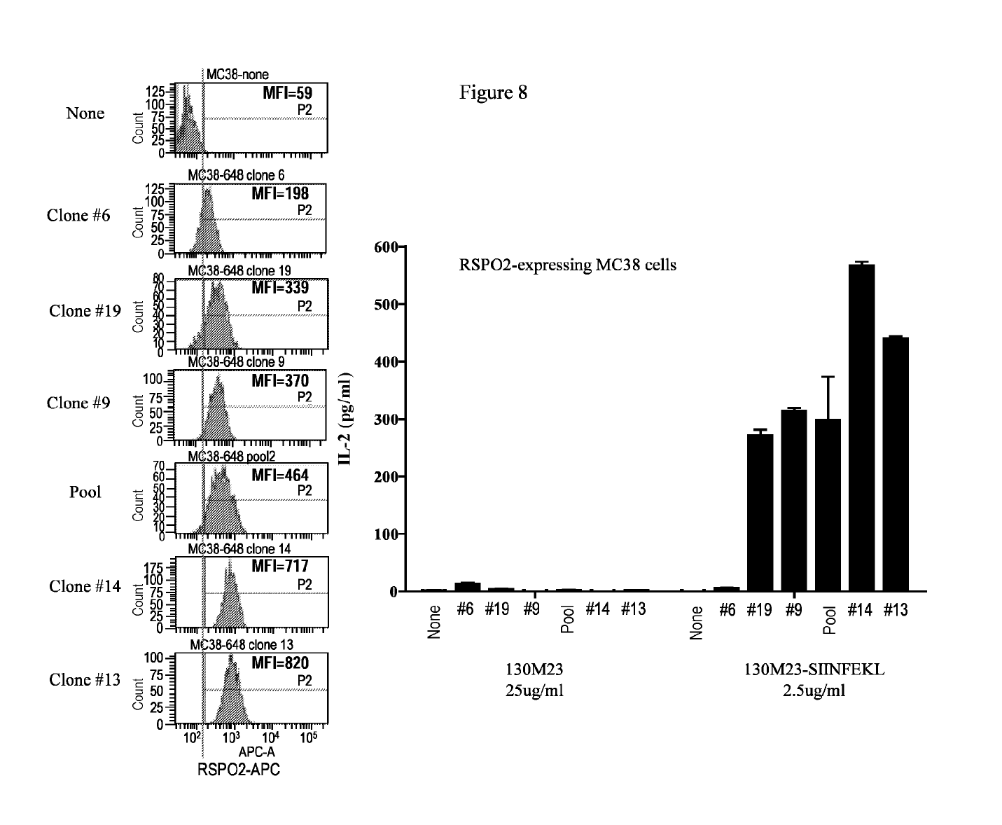

As shown in FIG. 8, cell surface expression of RSPO2 correlated well with the amount of IL-2 produced. These results demonstrate that the effect observed is target-specific.

### Example 7

### Cell Surface Expression of MHC Class I Antigens

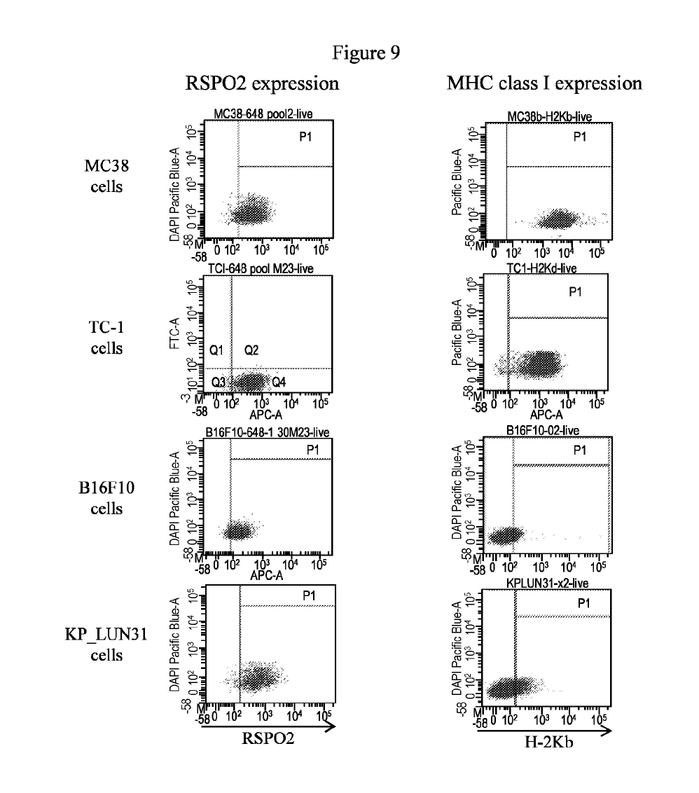

RSPO2-expressing MC38 and TC1 cells appeared to be more proficient at presenting the OVA antigen than the RSPO2-expressing B16F10 and KP_LUN31 cells. This was demonstrated by the low amounts of 130M23-SIINFEKL antibody needed to elicit IL-2 production in the MC38 and TC1 cells as compared to the B16F10 and KP_LUN31 cells, and in the high amounts of IL-2 induced using the MC38 and TC1 cells. The level of cell surface expression of RSPO2 was very similar for all four cell lines (see FIG. 9), so the expression level of MHC class I antigens on each cell line was studied.

MHC class I antigen expression was assessed using an anti-H-2 Kb antibody (BioLegend) and flow cytometry.

As shown in FIG. 9, the cell lines that appeared to be most proficient at presenting the antigenic peptide OVA (MC38 and TC1) were observed to have higher levels of MHC class I antigens than the other two cell lines (B16F10 and KP_LUN31). These results indicate that detectable levels or a threshold level of MHC class I antigens may be needed to elicit productive presentation of an antigenic peptide. In addition, these results may support the use of MHC class I expression on tumor cells as a potential biomarker.

### Example 8

Antigen Presentation after Targeted Antibody Binding

The proof-of-concept study outlined in Example 3 was modified to target the tumor-associated antigen B7-H4. The modified model system consisted of (a) cancer cells expressing mouse B7-H4 on the cell surface, (b) an anti-mouse B7-H4 antibody comprising the OVA peptide SIINFEKL, and (c) the B3Z T-cell line that specifically becomes activated when the OVA peptide SIINFEKL is presented in the context of a MHC class I molecule.

Stable MC38 and TC1 cell lines expressing mouse B7-H4 on the cell surface were generated. MC38 or TC1 cells (1.5-2×10⁵ cells/well) were seeded in a 6-well plate and infected with a lentivirus construct expressing mouse B7-H4 protein. The construct comprises a full length mouse B7-H4 protein linked to GFP and expressed from a CMV promoter. Cells were infected at a MOI of 0.625 and 8 μg/ml POLYBRENE was added during infection to increase transduction efficiency. 72 hrs after infection, cells were trypsinized, stained with an anti-B7-H4 antibody, and B7-H4 expression on the cell surface was detected by flow cytometry. B7-H4-positive cells were sorted using an ARIA 2 FACS instrument (BD Pharmingen). Single cell clones were propagated in culture to establish stable cell lines.

An anti-mouse B7-H4 antibody comprising the OVA peptide was generated from anti-mB7-H4 antibody 278M6 (IgG1). The 278M6 antibody was re-engineered as a deglycosylated IgG1 single-chain antibody with a linker between the light chain and the variable domain of the heavy chain. The OVA peptide SIINFEKL (SEQ ID NO:5) was embedded within the linker sequence and did not interfere with binding of the antibody to mouse B7-H4. This antibody is referred to herein as 278M6-SIINFEKL (or alternatively, as 278M26).

B7-H4-expressing MC38 tumor cells as well as parental cells (2-5×10⁴ cells/well) were plated in collagen-coated 96-well, flat-bottom plates. B7-H4-expressing TC1 tumor cells as well as parental cells (1×10⁴ cells/well) were plated in 96-well, flat-bottom plates. Seven hours after cell seeding, cells were incubated with anti-mB7-H4 antibody 278M6 (5 μg/ml), 278M6-SIINFEKL (5, 1, or 0.2 μg/ml for MC38 cells and 5 or in/nil for TC1 cells), or no antibody. Cells were incubated for 16 hrs at 37° C. to allow for antibody binding, internalization, processing, and presentation. Cells were washed and mildly fixed with 0.2% paraformaldehyde for 10 min at room temperature. B3Z T-cells (1×10⁵/well) were added to each well, the co-cultured cells were incubated for 48 hrs at 37° C., and supernatants were harvested and analyzed for production of IL-2 as described above. Each sample was done in triplicate and controls included medium only and B3Z cells only.

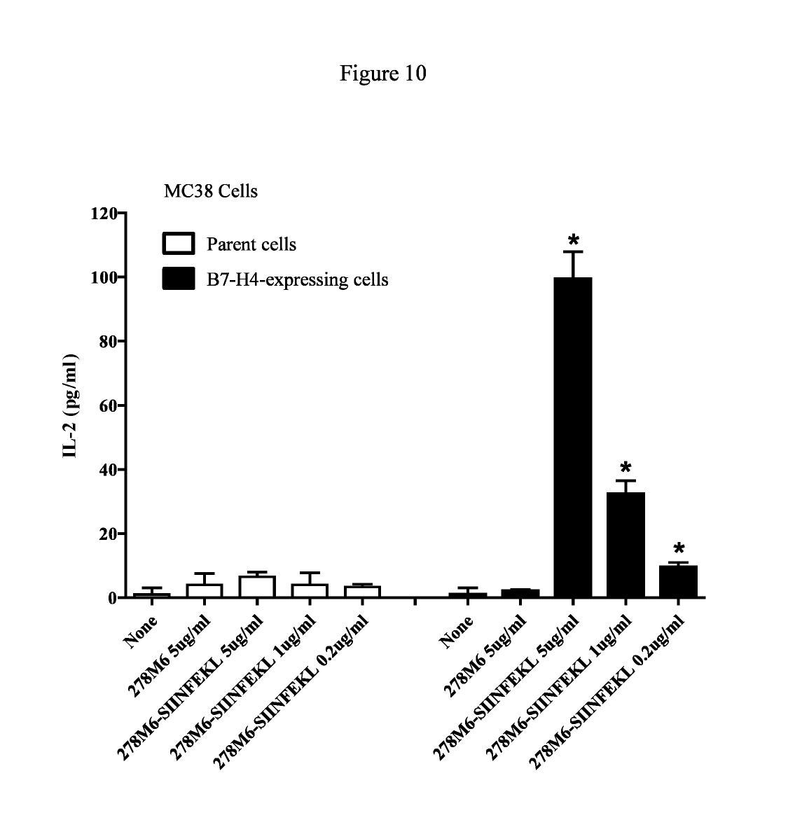

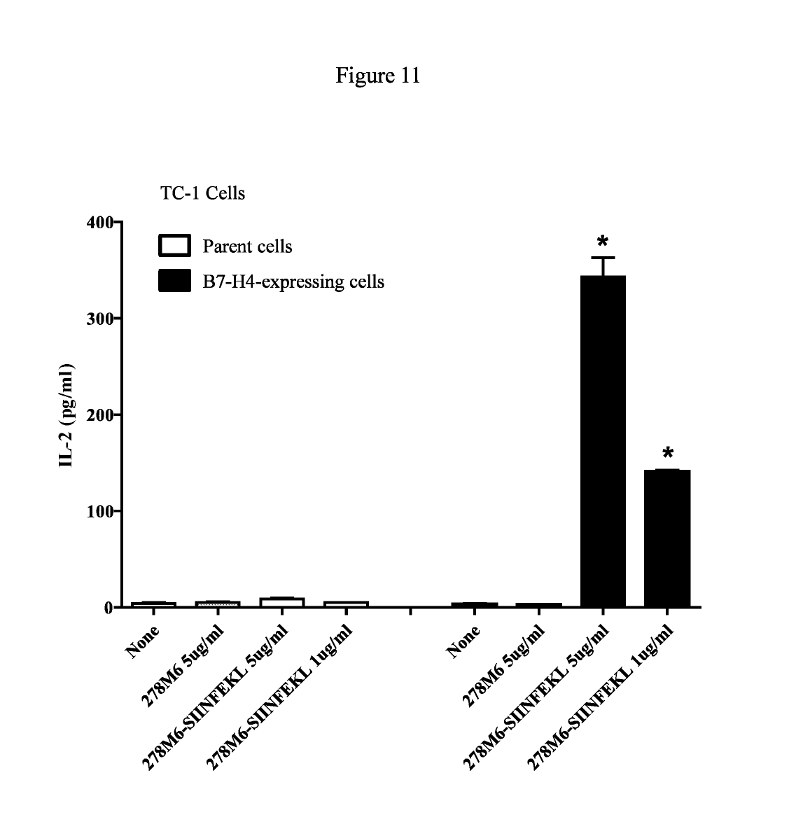

As shown in FIGS. 10 and 11, B7-H4-expressing MC38 (FIG. 10) and B7-H4-expressing TC1 (FIG. 11) cell lines were able to activate B3Z T-cells after binding of the 278M6-SIINFEKL antibody to its cell surface target. The production of IL-2 by the B3Z T-cells indicated that the B7-H4-expressing tumors had internalized the 278M6-SIINFEKL antibody, processed the OVA peptide SIINFEKL, and presented it on the cell surface in a proper MHC class I complex. As seen in the previous model, results were dependent on specific antibody recognition and binding to B7-H4 since the 278M6-SIINFEKL antibody elicited little or no IL-2 production from the parental cells. In addition, the 278M6 antibody (without an OVA peptide) did not activate the B3Z T-cells.

These data further demonstrate that tumor cells can successfully present antigenic peptides to T-cells via delivery of the antigenic peptide to the tumor cell by specific antibody-target binding.

### Example 9

Antigen Presentation after Targeted Antibody Binding in In Vivo Model

Adoptive cell transfer (ACT) experiments were set up to study delivery of an antigenic peptide to a tumor cell by specific antibody/target binding in an in vivo model. Activated T-cells from OT-I mice were prepared for adoptive T-cell transfer. Spleens were removed from OT-I transgenic mice and splenocytes were isolated. The cells were placed in tissue culture flasks (5×10⁶/ml in 10 mls) with OVA peptide SIINFEKL (1 μg/ml) in the presence of IL-2 (20 IU/ml) and incubated for 4 hours. After 4 hours, cells were spun in a centrifuge at 1000 rpm for 5 minutes and resuspended in 10 ml of media containing IL-2 (20 IU/ml) and incubated under standard conditions. On Days 1, 3, and 4, 30 ml of medium containing IL-2 was added. On Day 5, cells were collected, washed, and characterized by flow cytometry for CD3, CD4, CD8, CD44, CD62L, CD25, and CD69 expression using commercially available antibodies (Biolegend) and an ARIA 2 FACS instrument (BD Biosciences). To detect SIINFEKL-specific TCR on CD8+ cells, a H-2 Kb SIINFEKL-APC conjugated dextramer (Immudex) was used.

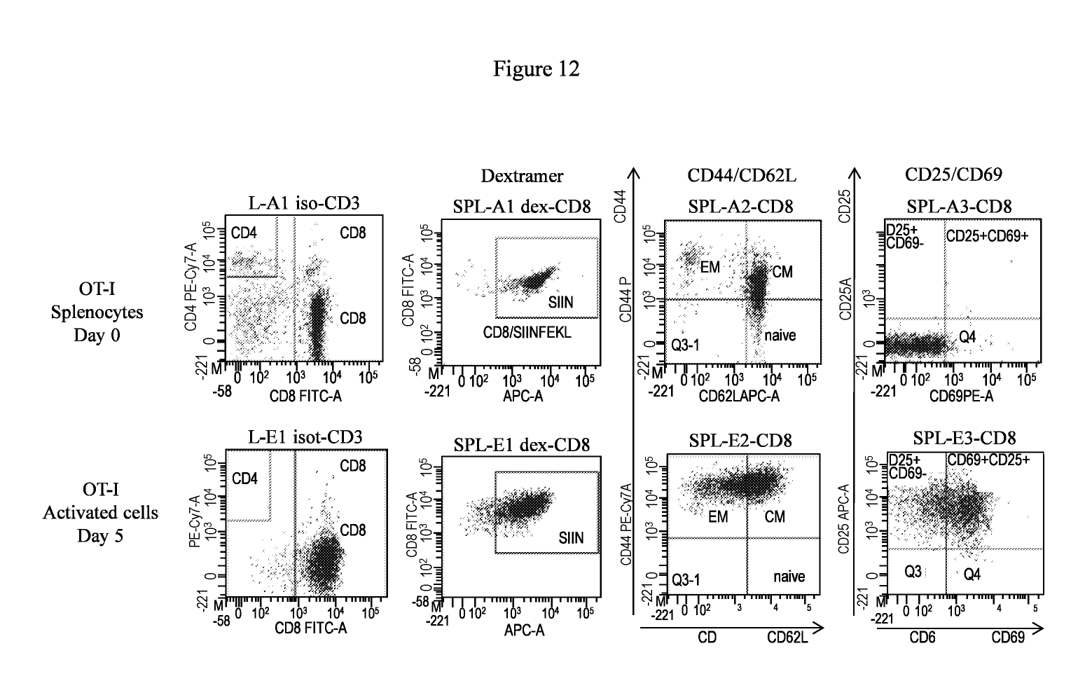

FIG. 12 shows the analysis of the OT-I activated cells. Five days after stimulation with OVA peptide SIINFEKL, T-cells were activated as demonstrated by high expression of CD25 on almost all cells and a significant cell population with high CD25/CD69. The cells were shown to be mostly effector and central memory cells as demonstrated by a population of CD44^{high}/CD62L^{low} (effector cells) and a population of CD44^{high}/CD62L^{high} (central memory cells). The cells were observed to be almost 100% CD3+/CD8+ T-cells. In addition, the activated cells were shown to be OVA peptide SIINFEKL-specific CD8+ T-cells as they were near 100% positive when stained with the H-2 Kb SIINFEKL dextramer.

The OT-I activated T-cells were tested in an adoptive cell transfer experiment using C57BL/6N mice. C57BL/6N mice were subcutaneously injected with E.G7-OVA cells (250,000 cells) or MC38-OVA tumor cells (500,000 cells) in 50% MATRIGEL. The murine E.G7-OVA and MC38-OVA cell lines synthesize and secrete ovalbumin constitutively. At Day 7, tumors were measured and mice were randomized (n=9-10 mice/group). On Day 7, mice were injected with OT-I activated T-cells (adoptive cell transfer) intravenously via tail vein in 200 ul at 1.8×10⁷ cells/mouse. Tumor growth was monitored and tumor volumes were measured with electronic calipers at the indicated time points.

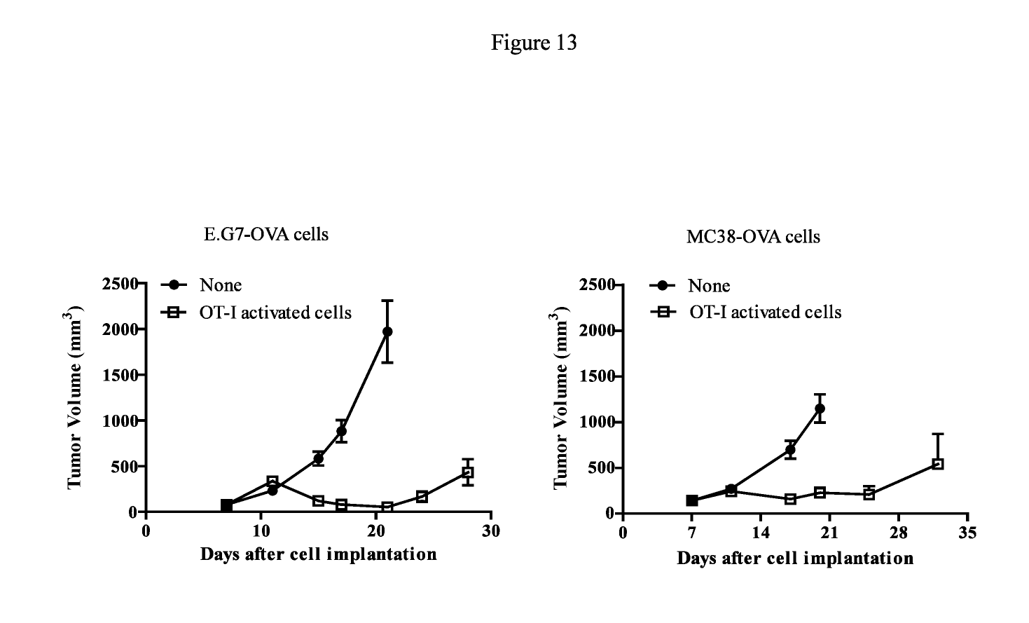

As shown in FIG. 13, the growth of E.G7-OVA tumors and MC38-OVA tumors was significantly inhibited by the presence of the transferred OT-I activated T-cells.

The OT-I activated T-cells were next tested in an adoptive cell transfer experiment using mice bearing RSPO2-expressing MC38 tumor cells and treated with the RSPO2-targeted antibody carrying the OVA peptide. C57BL/6 mice were subcutaneously injected with RSPO2-expressing MC38 cells or parental MC38 cells (500,000 cells) in 50% MATRIGEL. At Day 7, tumors were measured and mice were randomized (n=9-10 mice/group). Starting on Day 7, mice were treated with anti-RSPO2 antibody 130M23 or a deglycosylated version of antibody 130M23-SIINFEKL, (alternatively referred to as 130M30) at a dose of 15 mg/kg injected ip twice a week. On Day 8 the mice were injected with OT-I activated T-cells (adoptive cell transfer) intravenously via tail vein in 200 ul at 1.8×10⁷ cells/mouse. Tumor growth was monitored and tumor volumes were measured with electronic calipers at the indicated time points.

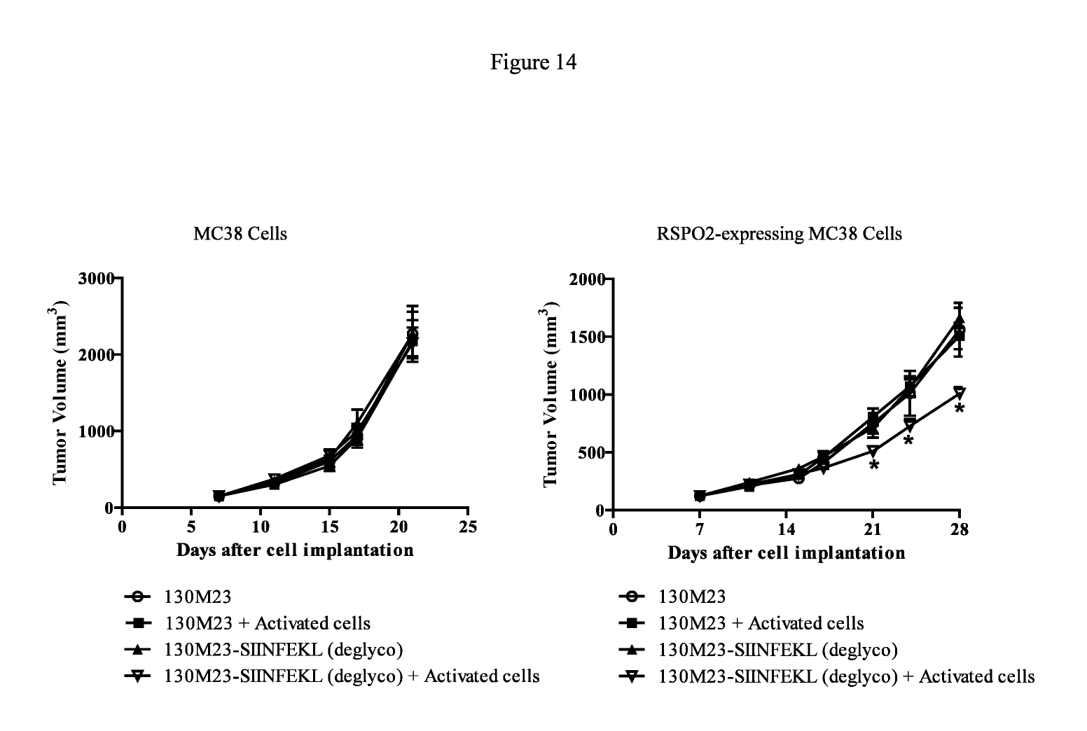

As shown in FIG. 14, the adoptive cell transfer of OT-I activated T-cells had no effect on tumor growth of the parental MC38 cells. In mice with RSPO2-expressing MC38 tumors, anti-RSPO2 antibody 130M23, with or without the presence of OT-I activated T-cells, had no effect on tumor growth. In addition, deglycosylated antibody 130M23-SIINFEKL had no effect on tumor growth in the absence of OVA-specific OT-I activated T-cells. In contrast, tumor growth in mice treated with deglycosylated antibody 130M23-SIINFEKL in the presence of OVA-specific OT-I activated T-cells was significantly inhibited.

Overall, these data demonstrate that antigen presentation can be achieved by tumor cells via antibody-receptor binding and delivery of a specific peptide antigen to the tumor cell by an antibody. Importantly, antigen presentation at the surface of the tumor cell can combine with a specific T-cell response and result in tumor growth inhibition in vivo.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

Following are the sequences disclosed in the application:

Human IgG1 Heavy chain constant region
(SEQ ID NO: 1)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region
(SEQ ID NO: 2)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region
(SEQ ID NO: 3)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 Heavy chain constant region
(SEQ ID NO: 4)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
OVA peptide
(SEQ ID NO: 5)
SIINFEKL
Human B7-H4 amino acid sequence with signal sequence
(SEQ ID NO: 6)
MASLGQILFWSIISIIIILAGAIALIIGFGISGRHSITVTTVASAGNIGEDGILSCTFEP
DIKLSDIVIQWLKEGVLGLVHEFKEGKDELSEQDEMFRGRTAVFADQVIVGNASLRLKNV
QLTDAGTYKCYIITSKGKGNANLEYKTGAFSMPEVNVDYNASSETLRCEAPRWFPQPTVV
WASQVDQGANFSEVSNTSFELNSENVTMKVVSVLYNVTINNTYSCMIENDIAKATGDIKV
TESEIKRRSHLQLLNSKASLCVSSFFAISWALLPLSPYLMLK
Human B7-H4 amino acid sequence without predicted signal sequence
(SEQ ID NO: 7)
LIIGFGISGRHSITVTTVASAGNIGEDGILSCTFEPDIKLSDIVIQWLKEGVLGLVHEFK
EGKDELSEQDEMFRGRTAVFADQVIVGNASLRLKNVQLTDAGTYKCYIITSKGKGNANLE
YKTGAFSMPEVNVDYNASSETLRCEAPRWFPQPTVVWASQVDQGANFSEVSNTSFELNSE
NVTMKVVSVLYNVTINNTYSCMIENDIAKATGDIKVTESEIKRRSHLQLLNSKASLCVSS
FFAISWALLPLSPYLMLK
Human B7-H4 Extracellular domain amino acid sequence
(SEQ ID NO: 8)
LIIGFGISGRHSITVTTVASAGNIGEDGILSCTFEPDIKLSDIVIQWLKEGVLGLVHEFK
EGKDELSEQDEMFRGRTAVFADQVIVGNASLRLKNVQLTDAGTYKCYIITSKGKGNANLE
YKTGAFSMPEVNVDYNASSETLRCEAPRWFPQPTVVWASQVDQGANFSEVSNTSFELNSE
NVTMKVVSVLYNVTINNTYSCMIENDIAKATGDIKVTESEIKRRSHLQLLNSKASL
278M1 Heavy chain CDR1
(SEQ ID NO: 9)
TSYYMH
278M1 Heavy chain CDR2
(SEQ ID NO: 10)
YVDPFNGGTSYNQKFKG
278M1 Heavy chain CDR3
(SEQ ID NO: 11)
FIAGFAN
278M1 Heavy chain CDR3
(SEQ ID NO: 12)
IAGFAN
278M1 Light chain CDR1
(SEQ ID NO: 13)
KASQDIKSYLS
278M1 Light chain CDR2
(SEQ ID NO: 14)
YATSLAD
278M1 Light chain CDR3
(SEQ ID NO: 15)
LQHGESPYT
278M1 Light chain CDR3
(SEQ ID NO: 16)
LQHGESPY
278M1 Heavy chain variable region
(SEQ ID NO: 17)
QVQLQQSGAELMKPGASVKISCKASDYSFTSYYMHWVKQSHGKSLEWVGYVDPFNGGTSYNQKFKGKA
TLTVDKSSSTAYMHLSSLTSEDSGVYYCAFIAGFANWGQGTLVTVSA
278M1 Light chain variable region
(SEQ ID NO: 18)
DIVMTQSPSSMYASLGERVTITCKASQDIKSYLSWYQQKPWKSPKTLIYYATSLADGVPSR
FSGSGSGQDFSLTISSLESDDTATYYCLQHGESPYTFGGGTKLEIK
278M1 Heavy chain amino acid sequence with signal sequence underlined
(SEQ ID NO: 19)
MKHLWFFLLLVAAPRWVLSQVQLQQSGAELMKPGASVKISCKASDYSFTSYYMHWVKQSHGKSLEWVG
YVDPFNGGTSYNQKFKGKATLTVDKSSSTAYMHLSSLTSEDSGVYYCAFIAGFANWGQGTLVTVSAAK
TTPPSVYPLAPGSAAQTNSMVTLGCLVKGYFPEPVTVTWNSGSLSSGVHTFPAVLQSDLYTLSSSVTV
PSSTWPSETVTCNVAHPASSTKVDKKIVPRDCGCKPCICTVPEVSSVFIFPPKPKDVLTITLTPKVTC
VVVDISKDDPEVQFSWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWNGQPAENYKNTQPI
MDTDGSYFVYSKLNVQKSNWEAGNTFTCSVLHEGLHNHHTEKSLSHSPGK
278M1 Heavy chain amino acid sequence without signal sequence
(SEQ ID NO: 20)
QVQLQQSGAELMKPGASVKISCKASDYSFTSYYMHWVKQSHGKSLEWVGYVDPFNGGTSYNQKFKGKA
TLTVDKSSSTAYMHLSSLTSEDSGVYYCAFIAGFANWGQGTLVTVSAAKTTPPSVYPLAPGSAAQTNS
MVTLGCLVKGYFPEPVTVTWNSGSLSSGVHTFPAVLQSDLYTLSSSVTVPSSTWPSETVTCNVAHPAS
STKVDKKIVPRDCGCKPCICTVPEVSSVFIFPPKPKDVLTITLTPKVTCVVVDISKDDPEVQFSWFVD
DVEVHTAQTQPREEQFNSTFRSVSELPIMHQDWLNGKEFKCRVNSAAFPAPIEKTISKTKGRPKAPQV
YTIPPPKEQMAKDKVSLTCMITDFFPEDITVEWQWNGQPAENYKNTQPIMDTDGSYFVYSKLNVQKSN
WEAGNTFTCSVLHEGLHNHHTEKSLSHSPGK
278M1 Light chain amino acid sequence with signal sequence underlined
(SEQ ID NO: 21)
MKHLWFFLLLVAAPRWVLSDIVMTQSPSSMYASLGERVTITCKASQDIKSYLSWYQQKPWKSPKTLIY
YATSLADGVPSRFSGSGSGQDFSLTISSLESDDTATYYCLQHGESPYTFGGGTKLEIKRADAAPTVSI
FPPSSEQLTSGGASVVCFLNNFYPKDINVKWKIDGSERQNGVLNSWTDQDSKDSTYSMSSTLTLTKDE
YERHNSYTCEATHKTSTSPIVKSFNRNEC
278M1 Light chain amino acid sequence without signal sequence
(SEQ ID NO: 22)
DIVMTQSPSSMYASLGERVTITCKASQDIKSYLSWYQQKPWKSPKTLIYYATSLADGVPSRFSGSGSG
QDFSLTISSLESDDTATYYCLQHGESPYTFGGGTKLEIKRADAAPTVSIFPPSSEQLTSGGASVVCFL
NNFYPKDINVKWKIDGSERQNGVLNSWTDQDSKDSTYSMSSTLTLTKDEYERHNSYTCEATHKTSTSP
IVKSFNRNEC

What is claimed is:

1. An antibody that specifically binds a tumor-associated antigen (TAA), wherein the antibody comprises an exogenous polypeptide comprising at least one antigenic peptide.

2. The antibody of claim 1, wherein the antigenic peptide comprises a T-cell epitope.

3. The antibody of claim 2, wherein the T-cell epitope is a CD8+ T-cell epitope.

4. The antibody of claim 2, wherein the T-cell epitope is a MHC Class I-restricted epitope.

5. The antibody of any one of claims 1-4, wherein the exogenous polypeptide is derived from a virus.

6. The antibody of claim 5, wherein the virus is selected from the group consisting of: measles virus, varicella-zoster virus, influenza virus, mumps virus, poliovirus, rubella virus, rotavirus, hepatitis A virus, hepatitis B virus, Epstein Barr virus, and cytomegalovirus.

7. The antibody of any one of claims 1-6, wherein the light chain of the antibody comprises the exogenous polypeptide.

8. The antibody of any one of claims 1-6, wherein the exogenous polypeptide is attached to the N-terminus of the light chain of the antibody.

9. The antibody of any one of claims 1-6, wherein the exogenous polypeptide is attached to the C-terminus of the light chain of the antibody.

10. The antibody of any one of claims 1-6, wherein the exogenous polypeptide is within the variable region of the light chain of the antibody.

11. The antibody of any one of claims 1-6, wherein the exogenous polypeptide is within a CDR of the light chain of the antibody.

12. The antibody of any one of claims 1-6, wherein the heavy chain of the antibody comprises the exogenous polypeptide.

13. The antibody of any one of claims 1-6, wherein the exogenous polypeptide is attached to the N-terminus of the heavy chain of the antibody.

14. The antibody of any one of claims 1-6, wherein the exogenous polypeptide is attached to the C-terminus of the heavy chain of the antibody.

15. The antibody of any one of claims 1-6, wherein the exogenous polypeptide is within the variable region of the heavy chain of the antibody.

16. The antibody of any one of claims 1-6, wherein the exogenous polypeptide is within a CDR of the heavy chain of the antibody.

17. The antibody of any one of claims 1-6, which is a single chain antibody wherein the heavy chain of the antibody is linked to the light chain of the antibody.

18. The antibody of claim 17, wherein the heavy chain and the light chain are linked by an amino acid sequence.

19. The antibody of claim 18, wherein the amino acid sequence between the heavy chain and the light chain comprises the exogenous polypeptide.

20. The antibody of any one of claims 1-19, wherein the TAA is selected from the group consisting of: B7-H4/VTCN1, B7-H3, HER2, CD47, DLL3, CD20, CEA, MUC16, STEAP2, FOLH1, STEAP1, SLC45A3, OXTR, CDH3, GABRP, ECEL1, SIGLEC11, and DIO3.

21. The antibody of any one of claims 1-19, wherein the TAA is selected from the group consisting of: ACPP, GPA33, GUGY2C, GARP, MUC-1, CEA, TERT, WT1, PSA, PSMA, PAP, PSCA, and TARP.

22. The antibody of any one of claims 1-19, wherein the TAA is B7-H4/VTCN1.

23. The antibody of claim 22, which comprises:
(a) a heavy chain CDR1 comprising TSYYMH (SEQ ID NO:9), a heavy chain CDR2 comprising YVDPFNGGTSYNQKFKG (SEQ ID NO:10), and a heavy chain CDR3 comprising FIAGFAN (SEQ ID NO:11) or IAGFAN (SEQ ID NO:12); and
(b) a light chain CDR1 comprising KASQDIKSYLS (SEQ ID NO:13), a light chain CDR2 comprising YATSLAD (SEQ ID NO:14), and a light chain CDR3 comprising LQHGESPYT (SEQ ID NO:15) or LQHGESPY (SEQ ID NO:16).

24. The antibody of any one of claims 1-23, which is a monoclonal antibody

25. The antibody of any one of claims 1-24, which is a humanized antibody or a human antibody.

26. The antibody of any one of claims 1-24, which is a recombinant antibody or a chimeric antibody.

27. The antibody of any one of claims 1-26, which is a bispecific antibody.

28. The antibody of any one of claims 1-23, which is an antibody fragment comprising an antigen binding site.

29. The antibody of any one of claims 1-27, which is an IgG antibody.

30. The antibody of claim 29, which is an IgG1 antibody, an IgG2 antibody, or an IgG4 antibody.

31. The antibody of any one of claims 1-30, which delivers the exogenous polypeptide to a tumor cell expressing the TAA.

32. The antibody of any one of claims 1-31, which is internalized by the tumor cell wherein an antigenic peptide is presented on the surface of the tumor cell.

33. The antibody of any one of claims 1-30, which delivers the exogenous polypeptide to a tumor cell and wherein an antigenic peptide is presented on the surface of the tumor cell.

34. The antibody of any one of claims 1-33, which inhibits tumor growth.

35. The antibody of any one of claims 1-34, which induces, increases, activates cytolytic T-cells.

36. The antibody of any one of claims 1-34, which induces, increases, and/or activates T-cell killing of tumor cells.

37. A cell comprising or producing the antibody of any one of claims 1-36.

38. A composition comprising the antibody of any one of claims 1-36.

39. A pharmaceutical composition comprising the antibody of any one of claims 1-36 and a pharmaceutically acceptable carrier.

40. An isolated polynucleotide molecule comprising a polynucleotide that encodes an antibody of any one of claims 1-36.

41. A vector comprising the polynucleotide of claim 40.

42. An isolated cell comprising the polynucleotide of claim 40.

43. An isolated cell comprising the vector of claim 41.

44. A method of increasing the immunogenicity of a tumor, the method comprising contacting tumor cells with an effective amount of an antibody of any one of claims 1-36.

45. A method of increasing the immunogenicity of a tumor in a subject, the method comprising administering to the subject a therapeutically effective amount of an antibody of any one of claims 1-36.

46. A method of redirecting an existing immune response to a TAA-expressing tumor, the method comprising administering to a subject a therapeutically effective amount of an antibody of any one of claims 1-36.

47. The method of claim 46, wherein the existing immune response is against a virus.

48. The method of claim 46 or claim 47, wherein the existing immune response is a cell-mediated response.

49. The method of any one of claims 46-48, wherein the existing immune response comprises cytotoxic T-cells.

50. A method of inhibiting growth of a tumor, the method comprising contacting the tumor with an effective amount of an antibody of any one of claims 1-36.

51. A method of inhibiting growth of a tumor in a subject, the method comprising administering to the subject a therapeutically effective amount of an antibody of any one of claims 1-36.

52. The method of any one of claims 44-51, wherein the tumor or tumor cell is selected from the group consisting of colorectal tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

53. A method of treating cancer in a subject, the method comprising administering to the subject a therapeutically effective amount of an antibody of any one of claims 1-36.

54. The method of claim 53, wherein the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

55. The method of any one of claims 45-54, which comprises administering at least one additional therapeutic agent.

56. The method of claim 55, wherein the additional therapeutic agent is a chemotherapeutic agent.

57. The method of claim 55, wherein the additional therapeutic agent is an antibody.

58. The method of claim 55, wherein the additional therapeutic agent is an immunotherapeutic agent.

59. The method of claim 58, wherein the immunotherapeutic agent is selected from the group consisting of: anti-CTLA-4 antibody, anti-CD28 antibody, anti-CD3 antibody, anti-PD-1 antibody, anti-PD-L1 antibody, anti-TIGIT antibody, an anti-GITR antibody, an anti-OX-40 antibody, an anti-CD40 antibody, or an anti-4-1BB antibody, TLR4, TLR7, TLR9, a soluble ligand such as GITRL, GITRL-Fc, OX-40L, OX-40L-Fc, CD40L, CD40L-Fc, 4-1BB ligand, or 4-1BB ligand-Fc.

60. The method of claim 55, wherein the additional therapeutic agent is an inhibitor of the Notch pathway, the Wnt pathway, or the RSPO/LGR pathway.

61. The method of any one of claims 45-49 and 51-60, wherein the subject has had a tumor or a cancer removed.

62. The method of any one of claims 45-49 and 51-60, wherein the subject has had a tumor or a cancer previously treated.
